(19) United States
(12) Patent Application Publication
(10) Pub. No.: US 2026/0026713 A1
(43) Pub. Date: Jan. 29, 2026

(54) MULTIMODALITY MOTION SENSING

(71) Applicant: HOFFMANN-LA ROCHE INC., Little Falls, NJ (US)

(72) Inventors: Mike Domenik RINDERKNECHT, Basel (CH); Dimitar Yuriev STANEV, Basel (CH); Mattia ZANON, Basel (CH)

(21) Appl. No.: 19/349,392

(22) Filed: Oct. 3, 2025

Related U.S. Application Data
(63) Continuation of application No. PCT/EP2024/059373, filed on Apr. 5, 2024.

(30) Foreign Application Priority Data
Apr. 5, 2023 (EP) 23166831.0

(51) Int. Cl. A61B 5/11; A61B 5/00; G16H 40/63

(52) U.S. Cl. CPC A61B 5/112; A61B 5/1121; A61B 5/1127; A61B 5/6823; G16H 40/63; A61B 2562/0219

(57) ABSTRACT

The invention provides methods comprising: (i) providing data from a wearable motion sensing system worn by a subject and data from a reference motion sensing system recording the movement of the subject; and (ii) synchronizing the data from the wearable motion sensing system and the reference motion sensing system by: determining a first signal derived from the acceleration and optionally the angular velocity of a sensor of the wearable motion sensing system, as a function of time; determining a corresponding second signal derived from the position of one or more markers as measured by the reference motion sensing system and/or the ground reaction force applied to feet of the subject, as a function of time; and identifying a time lag that aligns the first signal and the second signal. Related methods, systems and products are also described.

### CROSS REFERENCE TO RELATED APPLICATIONS

This application is a continuation of International Application No. PCT/EP2024/059373, filed on Apr. 5, 2024, which claims the benefit of EP application Ser. No. 23/166,831.0, filed Apr. 5, 2023, the disclosures of which are herein incorporated by reference in their entirety.

### FIELD OF INVENTION

The present invention relates to a method for analysing data from motion sensing systems comprising one or more wearable motion sensors and one or more reference motion sensing systems. It is particularly, but not exclusively, concerned with a method for synchronising data from wearable motion sensors and from motion marker tracking systems. Methods for analysing data from motion sensing systems, and method for analysing gait features of a subject using such methods are also described.

### BACKGROUND TO THE INVENTION

Smartphone sensor-based tests are gaining increasing attention for assessing functional impairment related to neurologic disorders such as multiple sclerosis (MS). For example, Montalban et al. [1] describe a smartphone app (the “Floodlight PoC app”) designed to assess functional abilities across three key domains affected by MS: cognition, upper extremity function, and gait and balance. In order to establish the clinical and analytical validity of smartphone-based gait and balance tests included in technologies such as the Floodlight app, a critical step is ensuring that the measures derived from these tests are accurate and precise. This includes comparing the sensor data collected with the Floodlight technology against data collected simultaneously with reference measurement systems (such as e.g., motion-capture systems or ground reaction forces (GRF) plates). More generally, in any situation where wearable motion sensing devices data is to be compared to reference measurement systems, several technical challenges arise. For example, it is often difficult to cross-reference the data from different measurement systems, ensuring that the data relates to the same instance of recording (e.g. test execution) and that it is synchronized. Solutions for synchronizing such data are available, but they either require the use of their application programming interface (API) (Kothe et al.), which may not be always feasible or desirable, or can result in small, persisting lags between measurement systems that are not fully accounted for (see e.g. TimeServer, www.timeservers.net/sync/ntp/android).

Therefore, there is a need for improved methods for jointly analysing motion sensing data from a plurality of different measurement systems.

### STATEMENTS OF INVENTION

The present inventors identified at least two important challenges with analysis of motion sensing data from different measurement systems. One is to guarantee that the information on test type (i.e., test identifier and test condition) has been correctly entered on the different measurement systems. This ensures that subsequent analyses only compare data collected during the same test execution. A second challenge is to accurately synchronize sensor data collected with the different systems. They therefore devised solutions for both validating metadata such as test type and for synchronizing sensor data. They illustrated their effectiveness using data obtained with the Floodlight technology and a motion-capture system.

The present disclosure provides computer-implemented methods comprising:
(i) providing data from a wearable motion sensing system worn by a subject and data from a reference motion sensing system recording the movement of the subject, wherein the reference motion sensing system is configured to record the position of one or more markers as a function of time and/or the ground reaction force applied to the feet of the subject as a function of time, and wherein the wearable motion sensing system is configured to record the acceleration and optionally the angular velocity of one or more wearable motion sensors comprised in the wearable motion sensing system worn by the subject; and
(ii) synchronizing the data from the wearable motion sensing system and the reference motion sensing system by:
   determining a first signal derived from the acceleration and optionally the angular velocity of a sensor of the wearable motion sensing system, as a function of time;
   determining a corresponding second signal derived from the position of one or more markers as measured by the reference motion sensing system and/or the ground reaction force applied to feet of the subject, as a function of time; and
   identifying a time lag that aligns the first signal and the second signal.

In embodiments of the general aspect above, the reference motion sensing system is configured to record the ground reaction force applied to the feet of the subject as a function of time. In embodiments of this aspect, the first signal may be derived from the acceleration of a sensor of the wearable motion sensing system (as measured by the sensor), and the corresponding second signal may be derived from the ground reaction force applied to a foot or feet of the subject (as measured by the reference motion sensing system). Embodiments of this aspect may have any of the features described in relation to the second aspect below.

In other embodiments of the general aspect above, the reference motion sensing system is configured to record the position of one or more markers as a function of time. In embodiments of this aspect, the first signal may be derived from the acceleration and the angular velocity of a sensor of the wearable motion system (as measured by the sensor), and the corresponding second signal may be derived from the position of one or more markers as measured by the reference motion sensing system. In embodiments of this aspect the first signal and the second signal may both be determined in a first reference frame. Embodiments of this aspect may have any of the features described in relation to the third aspect below.

Advantageously, the methods described herein only use the raw signals from the various sensors to synchronise their signals, by deriving, from said raw signals, derived signals that are comparable between the sensors. Indeed, the present inventors realised that although the quantities measures by the different sensors differ and are therefore not directly comparable, it is possible to use these measurements to obtain derived signals that are comparable (e.g. because they represent the same or correlated physical metrics). As a result, the methods described herein are applicable to any wearable motions sensor device and any reference motion system regardless of their interfaces, the software they work with, etc. For example, the methods can be applied to synchronise any mocap system with any IMU, even if the mocap system and IMU were not provided with dedicated firmware for their synchronisation (or a common clock). The methods described herein therefore have full flexibility. This is also an advantage of the methods for synchronisation of multiple devices by mechanical perturbation described herein, which do not rely on compatible software in the different sensors.

Thus, according to a second aspect, the disclosure provides a computer-implemented method comprising: providing data from a wearable motion sensing system worn by a subject and data from a reference motion sensing system recording the movement of the subject, wherein the reference motion sensing system is configured to record the ground reaction force applied to the feet of the subject as a function of time and wherein the wearable motion sensing system is configured to record the acceleration of one or more wearable motion sensors comprised in the wearable motion sensing system worn by the subject; and synchronizing the data from the wearable motion sensing system and the reference motion sensing system by: determining a first signal using the vector magnitude of the acceleration recorded by a sensor of the wearable motion sensing system as a function of time, determining a second signal using the vector magnitude of a force applied to a foot or feet of the subject recorded by the reference motion sensing system as a function of time; and identifying a time lag that aligns the first signal and the second signal.

The method may have any one or more of the following optional features.

The sensor used to determine the first signal may be a wearable motion sensor located on the subject. The sensor used to determine the first signal may be a wearable motion sensor located proximal to the centre of mass of the subject. The sensor used to determine the first signal may be a wearable motion sensor worn by the subject at the level of their waist. The wearable motion sensing system may comprise a plurality of wearable motion sensors and the sensor used to determine the first signal may be the wearable motion sensor of the plurality of wearable motion sensors that is located closest to the subject's centre of mass.

The wearable motion sensing system may comprise one or more wearable motion sensors that each comprise an accelerometer and optionally a gyroscope. The reference motion sensing system may comprise one or more ground reaction force sensors. The one or more ground reaction force sensors may be multiaxial force plates.

Determining the second signal may comprise obtaining ∥f∥ where f is the ground reaction force measured by a sensor of the reference motion sensing system recording the ground reaction force on a foot of the subject as the subject steps onto said foot. Determining the second signal may comprise obtaining: ∥f₁∥+∥f₂∥ wherein f₁ and f₂ are the ground reaction forces measured by a first sensor and second sensors of the reference motion sensing system recording the ground reaction force on a first and second foot of the subject, respectively. Determining the second signal may comprise normalising the obtained values. Determining the first signal may comprise obtaining the vector magnitude of the acceleration recorded by a sensor of the wearable motion sensing system as a function of time. Determining the first signal may comprise normalising the obtained values (∥a∥). Normalising the obtained values (for the first and/or second signals) may comprise normalising each instance of the obtained value by dividing it by the maximum instance of the obtained values. Normalising the obtained values may comprise obtaining a z-score for each instance of the obtained values using the distribution of said obtained values.

The methods of the present aspect can comprise the steps of any embodiment of any other aspect described herein, such as e.g., the third aspect described below. For example, the reference motion system can be further configured to record the position of one or more markers as a function of time and synchronising the data from the wearable motion sensing system and the reference motion sensing system according to the present aspect can further comprise determining a first signal corresponding to the unit gravity direction associated with a sensor of the wearable motion system or to the acceleration of a sensor of the wearable motion sensing system adjusted to remove the contribution of the acceleration of gravity, as detected by the sensor of the wearable motion system, as a function of time and in a first reference frame; determining a second signal corresponding to the unit gravity direction or the acceleration of a marker associated with the sensor of the wearable motion system as detected by the reference motion sensing system, as a function of time and in the first reference frame; and identifying a time lag that aligns the first signal and the second signal. Indeed, it may be advantageous to synchronise the data from the wearable motion sensing system and the reference motion sensing system using a plurality of the methods described herein. In such embodiments, the synchronization method that results in the highest similarity (e.g. as assessed by visual alignment) between one or more motion features derived from the data from the wearable motion sensing system and corresponding features derived from the data from the reference motion sensing system may be selected to synchronise the data before further analysis.

According to a third aspect, there is provided a computer-implemented method comprising: providing data from a wearable motion sensing system worn by a subject and data from a reference motion sensing system recording the movement of the subject, wherein the reference motion sensing system is configured to record the position of one or more markers as a function of time and wherein the wearable motion sensing system is configured to record the acceleration and angular velocity of one or more wearable motion sensors comprised in the wearable motion sensing system worn by the subject; and synchronizing the data from the wearable motion sensing system and the reference motion sensing system by: determining a first signal derived from the acceleration and angular velocity of a sensor of the wearable motion sensing system, as a function of time; determining a corresponding second signal derived from the position of one or more markers as measured by the reference motion sensing system, as a function of time; and identifying a time lag that aligns the first signal and the second signal.

The method may have any one or more of the following optional features.

The first signal and second signal may both be determined in a first reference frame. The first reference frame may be the local reference frame of the sensor of the wearable motion sensing system (L). The first reference frame may be the reference frame of the reference motion sensing system (M).

A corresponding signal may be a signal that measures or approximates the same quantity as the first signal. For example, the first signal may correspond to the unit gravity direction associated with a sensor of the wearable motion system as a function of time and in a first reference frame, and the second signal may correspond to the unit gravity direction as detected by the reference motion sensing system, as a function of time and in the first reference frame. As another example, the first signal may correspond to the acceleration of a sensor of the wearable motion sensing system (as measured by the sensor), which can be either a linear acceleration or an acceleration that has been adjusted for gravity and the second signal may correspond to the acceleration of a marker associated with the sensor of the wearable motion system as detected by the reference motion sensing system, as a function of time and in the first reference frame.

The wearable motion sensing system may comprise one or more wearable motion sensors that each comprise an accelerometer and optionally a gyroscope. The reference motion sensing system may comprise a plurality of cameras located at different angles relative to the subject. The cameras may be infrared cameras.

The sensor of the wearable motion sensing system may be associated with a plurality of markers detectable by the reference motion sensing system, and the method may comprise identifying the value of a first rotation matrix (R_{LM}, R_{ML}) as a function of time, wherein the first rotation matrix aligns the reference frames of the sensor of the wearable motion system (L) and the reference motion system (M), and wherein identifying the value of the first rotation matrix as a function of time comprises identifying an orthogonal vector basis associated with the sensor in the reference frame of the reference motion sensing system using the plurality of markers. A new orthogonal vector basis may be identified at each time point for which a first and second signals are determined.

According to any embodiment of any aspect, an orthogonal vector basis may comprise three orthogonal vectors (e.g. an x, y and z axes).

The plurality of markers may be positioned so as to not be colinear. The plurality of markers may be positioned such that a first and second orthogonal vectors are created between the first and second and the second and third markers, respectively. A third orthogonal vector can be obtained from this using a cross-product. Thus, the plurality of markers detectable by the reference motion sensing system associated with the sensor of the wearable motion sensing system may comprise at least 3 markers that are not colinear. The at least 3 markers may be located on the sensor. Identifying an orthogonal vector basis associated with the sensor in the reference frame of the reference motion sensing system may use the 3 non-colinear markers.

The first rotation matrix may align the reference frame of the sensor of the wearable motion system (L) with the reference frame of the reference motion system (M). This may be referred to as rotation matrix R_{LM}. The first rotation matrix may align the reference frame of the reference motion system (M) with the reference frame of the sensor of the wearable motion system (L). This may be referred to as rotation matrix R_{ML}.

Synchronizing the data from the wearable motion sensing system and the reference motion sensing system may comprise: determining a first signal (g_{smartphoneL}(t)) corresponding to the unit gravity direction associated with a sensor of the wearable motion sensing system, as a function of time and in a first reference frame; determining a second signal (g_{mocapL}(t)) corresponding to the unit gravity direction measured by the reference motion sensing system, as a function of time and in the first reference frame; and identifying a time lag that aligns the first signal and the second signal. The first reference frame may be the local reference frame of the sensor of the wearable motion sensing system (L) and determining the second signal may comprise identifying the value of a first rotation matrix that aligns the reference frame of the reference motion sensing system with the first reference frame (R_{ML}), as a function of time. Alternatively, the first reference frame may be the reference frame of the reference motion sensing system (M), and determining the first signal comprises identifying the value of a first rotation matrix that aligns the local reference frame of the sensor with the first reference frame (R_{LM}), as a function of time. Determining the first signal may further comprise identifying the value of a second rotation matrix that aligns an axis of the local reference frame of the sensor with the gravitational axis (R_{GL}), as a function of time. Identifying the value of the second rotation matrix can comprise applying an orientation filter to the data (e.g. accelerometer and gyroscope data) from the sensor. The orientation filter may be a Madgwick filter. Determining the second signal may comprise determining the value of the expression R_{LM}(t)*

$${[ {v}_{1 M} , {v}_{2 M} , {v}_{3 M} ]}_{M}^{T}$$

where R_{LM}(t) is the value of a first rotation matrix that aligns the reference frame of the reference motion sensing system with the first reference frame, as a function of time, and [v_{1M},v_{2M},v_{3M}]_{M} is the unit gravity vector in the reference frame of the reference motion sensing system. The value of the vector [v_{1M},v_{2M},v_{3M}]_{M} depends on the conventions used by the reference motion system. One of [v_{1M},v_{2M},v_{3M}]_{M} may have the value of −1 and the other two may have the value of 0, the particular component having the value of −1 depending on the conventions used by the reference motion system. Determining the first signal may comprise determining the value of the expression

$${R}_{GL}^{T} ( t ) * {[ {v}_{1 G} , {v}_{2 G} , {v}_{3 G} ]}_{G}^{T}$$

where R_{GL}(t) is the value of a second rotation matrix that aligns an axis of the local reference frame of the sensor with the gravitational axis (R_{GL}), as a function of time, and [v_{1G},v_{2G},v_{3G}]_{G} is the unit gravity vector in the local reference frame of the sensor rotated to have an axis aligned with the gravitational axis. The value of the vector [v_{1G},v_{2G},v_{3G}]_{G} may depend on the axis that is being selected as the axis that is aligned to gravity in the local reference frame of the sensor rotated to have an axis aligned with the gravitational axis. One of [v_{1G},v_{2G},v_{3G}]_{M} may have the value of 1 and the other two may have the value of 0. Which particular axis is aligned with gravity and has the value of 1 may depends on the parameters of the procedure used to define the local reference frame of the sensor rotated to have an axis aligned with the gravitational axis.

Synchronizing the data from the wearable motion sensing system and the reference motion sensing system may comprise: determining a first signal corresponding to the acceleration of a sensor of the wearable motion sensing system, as measured by the sensor and adjusted to remove the contribution of the acceleration of gravity, as a function of time and in a first reference frame; determining a second signal corresponding to the acceleration of a marker associated with the sensor the wearable motion system as detected by the reference motion sensing system, as a function of time and in the first reference frame; and identifying a time lag that aligns the first signal and the second signal. Determining the second signal may comprise determining the position of the marker as a function of time in the first reference frame and determining the second derivative of the determined position. Determining the second signal may comprise smoothing the position of the marker as a function of time. Smoothing the position of the marker as a function of time may comprise filtering the signal (position of the marker as a function of time) to remove high-frequency signals. Smoothing the position of the marker as a function of time may comprise filtering the signal using a low-pass filter. Any low-pass filter known in the art may be used. Advantageously, the low pass filter can be a Butterworth filter. A low-pass filter may be associated with a predetermined passband, such as e.g. below 6 Hz, 7 Hz, 8 Hz or 9 Hz.

Smoothing the position of the marker as a function of time may comprise applying a smoothing spline to the position of the marker as a function of time. A smoothing spline may be fitted using generalised cross-validation. Determining the second signal may comprise using a Kalman filter to quantify noise in the position of the marker as a function of time and identify the first and second derivatives of the position of the marker as a function of time. The first reference frame may be the reference frame of the reference motion sensing system (M) and determining the position may comprise using the position of the marker recorded by the reference motion sensing system. Determining the first signal may comprise: identifying the value of a first rotation matrix (R_{LM}) as a function of time, wherein the first rotation matrix aligns the reference frame of the sensor of the wearable motion system (L) with the reference frame of the reference motion system (M), and transforming the acceleration of the sensor of the wearable motion sensing system into the first reference frame using the first rotation matrix. Determining the first signal may comprise removing the contribution of gravity from the transformed acceleration. Determining the first signal may comprise: obtaining a linear acceleration from the sensor, the first signal may be the magnitude of the linear acceleration from the sensor and the second signal may be the magnitude of the second derivative of the determined position of a marker associated with the sensor as measured by the reference motion sensing system. As explained above, the sensor of the wearable motion sensing system may be associated with a plurality of markers detectable by the reference motion sensing system and identifying the value of the first rotation matrix as a function of time may comprise identifying an orthogonal vector basis associated with the sensor in the reference frame of the reference motion sensing system using the plurality of markers. The plurality of markers detectable by the reference motion sensing system associated with the sensor of the wearable motion sensing system may comprise at least 3 markers that are not collinear. The at least 3 markers may be located on the sensor. Identifying an orthogonal vector basis associated with the sensor in the reference frame of the reference motion sensing system may use the 3 non-colinear markers. In embodiments where the wearable motion sensor measures linear acceleration of the sensor, the magnitude of this can be directly compared to the magnitude of the acceleration calculated from the position of a marker on the wearable motion sensor as measured by the reference motion sensing system. This is because acceleration magnitudes are invariant of the coordinate system, and the linear acceleration does not comprise a contribution of gravity. In embodiments where the wearable motion sensor measures an acceleration of the sensor that comprises a contribution of gravity, this acceleration may be transformed into the reference frame of the reference motion system, where the gravity direction is known and the acceleration can therefore be adjusted to remove the contribution of gravity. The magnitude of such an adjusted acceleration can thereafter be compared to the magnitude of the acceleration calculated from the position of a marker on the wearable motion sensor as measured by the reference motion sensing system. When the two accelerations are expressed in the same coordinate system (e.g. where the acceleration from the wearable motion sensor has been transformed into the reference frame of the reference motion sensing system), any component of the accelerations (e.g. any of the x, y or z components) can be compared.

The methods of the present aspect can comprise the steps of any embodiment of any other aspect described herein, such as e.g. the first or second aspects described above. For example, the reference motion sensing system may be further configured to record the ground reaction force applied to the feet of the subject as a function of time and synchronizing the data from the wearable motion sensing system and the reference motion sensing system may comprise: determining a first signal using the vector magnitude of the acceleration recorded by a sensor of the wearable motion sensing system as a function of time, determining a second signal as the sum of the vector magnitude of the forces applied to the feet of the subject recorded by the reference motion sensing system as a function of time; and identifying a time lag that aligns the first signal and the second signal. Indeed, it may be advantageous to synchronise the data from the wearable motion sensing system and the reference motion sensing system using a plurality of the methods described herein. In such embodiments, the synchronization method that results in the highest similarity between one or more motion features derived from the data from the wearable motion sensing system and corresponding features derived from the data from the reference motion sensing system may be selected to synchronise the data before further analysis.

The methods of any of the first, second or third aspects may have any one or more of the following optional features.

Identifying a time lag that aligns the first signal and the second signal may be performed by determining the cross-correlation of the first and second signals. Synchronizing the data from the wearable motion sensing system and the reference motion sensing system may comprise identifying a time lag that aligns the first signal and the second signals as the time lag corresponding to maximum of the cross-correlation of the first and second signal. Synchronizing the data from the wearable motion sensing system and the reference motion sensing system may comprise applying the identified time lag to the data from the wearable motion sensing system or the reference motion sensing system.

The method may further comprise resampling (e.g. downsampling and/or upsampling) the data from the wearable motion sensing system and/or the reference motion sensing system to a common sampling frequency. The common sampling frequency may be the highest common sampling frequency (of the frequencies in the data from the wearable motion sensing system and the reference motion sensing system).

Identifying or computing values “as a function of time” refers to identifying or computing values for a plurality of time points, to obtain a time dependent signal (i.e. a set of values with associated times at which these values were determined).

The wearable motion sensing system may comprise a plurality of wearable motion sensors and the method may further comprise synchronising the plurality of wearable motion sensors based on a signal corresponding to a mechanical perturbation to which the plurality of wearable motion sensors were simultaneously exposed, in data series recorded by the plurality of wearable motion sensors. The method may further comprise simultaneously exposing the plurality of wearable motion sensors to the mechanical perturbation. The method may further comprise identifying a portion of a data series recorded by each of the sensors that comprises the signal corresponding to the mechanical perturbation. Synchronising the plurality of wearable motion sensors may comprise selecting a sensor of the plurality of wearable motion sensors as a reference sensor and identifying a time lag for a sensor of the plurality of wearable motion sensor, optionally for each of the sensors individually, by aligning the data series of the sensor or portion thereof with the data series of the reference motion sensor or a portion thereof. Identifying a time lag by aligning the data series may comprise performing a cross-correlation between the data series.

The signal corresponding to a mechanical perturbation may comprise a signal at a first time point and a second time point, and the synchronising the plurality of wearable motion sensors may comprise: identifying a first time lag for a sensor of the plurality of wearable motion sensor, optionally for each of the sensors individually, by aligning a portion of the data series of the sensor comprising the signal at the first time point with a portion of the data series of the reference motion sensor comprising the signal at the first time point; and identifying a second time lag for a sensor of the plurality of wearable motion sensor, optionally for each of the sensors individually, by aligning a portion of the data series of the sensor comprising the signal at the second time point with a portion of the data series of the reference motion sensor comprising the signal at the second time point. Identifying a time lag by aligning the portions of the data series may comprise performing a cross-correlation between the portions of the data series. Synchronising can comprise adjusting the time stamps of a sensor to synchronise the sensor with a reference sensor using time lag(s). Adjusting the time stamps of a sensor to synchronise the sensor with a reference sensor using time lag(s) can comprise using the time lags to determine the parameters of a monotonic function applied to the time stamps of the sensor that applies the first time lag at the first time point and the second time lag at the second time point.

The method may further comprise synchronising a sensor of the wearable motion system with the reference motion sensing system based on a signal corresponding to a mechanical perturbation to which the sensor was exposed in the data recorded by the sensor, wherein the signal is associated with a predetermined time in the reference motion sensing system. The method may further comprise exposing the sensor of the wearable motion system to the mechanical perturbation at the predetermined time. The predetermined time may be the start of the recording from the reference motion sensing system. The method may comprise identifying a portion of a data series recorded by the sensor of the wearable motion system that comprises a signal corresponding to the mechanical perturbation. The method may comprise detecting the onset of the signal corresponding to the mechanical perturbation in the data recorded by the sensor. The method may comprise correcting a data series recorded by a sensor of the wearable motion system or a data series recorded by the reference motion sensing system using the time stamp of the identified onset or a difference between the time stamp of the identified onset and the time stamp of the predetermined time.

The start of the recording from the reference motion sensing system can be identified as the start of a data series from the reference motion sensing system. The method may comprise identifying a portion of a data series recorded by the sensor of the wearable motion system that comprises a signal corresponding to the mechanical perturbation. For example, identifying a portion of the data series may comprise identifying a portion of the data series that comprises one or more peaks above a first predetermined threshold and optionally below a second predetermined threshold in the data from the sensor (e.g. accelerometer data). The portion of the data series may start at or at a predetermined time before a detected onset of the first peak detected above a first predetermined threshold and optionally below a second predetermined threshold. The portion of the data series may finish at or at a predetermined time after last peak detected above a first predetermined threshold and optionally below a second predetermined threshold. Identifying a portion of a data series recorded by the sensor of the wearable motion system that comprises a signal corresponding to the mechanical perturbation may comprise searching a data series recorded by the sensor of the wearable motion system for a signal that satisfies one or more criteria (such as e.g. a signal above a predetermined magnitude). The searching may be performed within a predetermined window of time. Detecting the onset of the signal corresponding to the mechanical perturbation in the data recorded by the sensor may comprise detecting the earliest time stamp at which a data series recorded by the sensor (or a portion thereof) is above a predetermined threshold, and identifying the first local minimum that precedes said time stamp as the onset of the signal corresponding to the mechanical perturbation in the data recorded by the sensor.

The method may further comprise decoding metadata encoded in the data recorded by a sensor of the wearable motion sensing system using a signal in the data corresponding to a mechanical perturbation to which the sensor was exposed. The mechanical perturbation may be the same mechanical perturbation used to synchronise the sensor of the wearable motion system with the reference motion sensing system according to the above embodiments. In other words, the mechanical perturbation that is associated with a predetermined time in the reference motion sensing system may also be configured to encode metadata in the data recorded by the sensor of the wearable motion sensing device. In embodiments, the mechanical perturbation is configured to generate a signal in the data of the sensor that is exposed to the mechanical perturbation, the signal having a predetermined pattern that can be detected and associated with corresponding metadata. The method may comprise detecting a predetermined pattern in the data recorded by the sensor and identifying metadata associated with the predetermined pattern.

The method may comprise exposing the sensor of the wearable motion system to the mechanical perturbation (optionally at a predetermined time, which may be the start of the recording from the reference motion sensing system). The method may comprise identifying a portion of a data series recorded by the sensor of the wearable motion system that comprises a signal corresponding to the mechanical perturbation. For example, identifying a portion of the data series may comprise identifying a portion of the data series that comprises one or more peaks above a first predetermined threshold and optionally below a second predetermined threshold in the data from the sensor (e.g. accelerometer data). The portion of the data series may start at or at a predetermined time before a detected onset of the first peak detected above a first predetermined threshold and optionally below a second predetermined threshold. The portion of the data series may finish at or at a predetermined time after last peak detected above a first predetermined threshold and optionally below a second predetermined threshold. The method may comprise detecting the onset of the signal corresponding to the mechanical perturbation in the data recorded by the sensor.

The predetermined pattern may comprise one or more peaks that satisfy one or more predetermined criteria. The method may comprise determining whether a peak that satisfies the one or more predetermined criteria is present at each of a plurality of time points, wherein at each of the plurality of time points, the presence of a peak that satisfies the one or more predetermined criteria is interpreted as a first value of a binary representation and the absence of a peak that satisfies the one or more predetermined criteria is interpreted as a second value of the binary representation. The plurality of time points may be a plurality of integer multiples of a predetermined time period from a detected onset of the predetermined pattern. The one or more predetermined criteria may be criteria applying to the height and/or shapes of the peaks and/or the distances between a plurality of peaks. For example, a predetermined criterion may be selected from: the peak having a height above a first predetermined value, the peak being a peak with a single local maximum, the peak being a peak without a shoulder, the peak being a local maximum with a height above a predetermined value, the peak being separated from the nearest peak by a distance above a predetermined value, etc. The method of may comprise obtaining the metadata by searching a database or memory storing metadata in association with a respective predetermined pattern in the signal of the sensor and/or a binary code derived therefrom.

According to a fourth aspect, there is provided a method comprising: (i) providing, to a processor, data from a wearable motion sensing system worn by a subject and data from a reference motion sensing system recording the movement of the subject, wherein the reference motion sensing system is configured to record the position of one or more markers as a function of time and/or the ground reaction force applied to the feet of the subject as a function of time, and wherein the wearable motion sensing system is configured to record the acceleration and optionally the angular velocity of one or more wearable motion sensors comprised in the wearable motion sensing system worn by the subject; and (ii) synchronizing, by said processor, the data from a sensor of the wearable motion system with the reference motion sensing system based on a signal corresponding to a mechanical perturbation to which the sensor was exposed in the data recorded by the sensor, wherein the signal is associated with a predetermined time in the reference motion sensing system.

The method according to the present aspect may have any one or more of the following optional features. The method may comprise exposing the sensor of the wearable motion system to the mechanical perturbation at the predetermined time. The predetermined time may be the start of the recording from the reference motion sensing system. The method may comprise identifying a portion of a data series recorded by the sensor of the wearable motion system that comprises a signal corresponding to the mechanical perturbation. The method may comprise detecting the onset of the signal corresponding to the mechanical perturbation in the data recorded by the sensor. The method may comprise correcting a data series recorded by a sensor of the wearable motion system or a data series recorded by the reference motion sensing system using the time stamp of the identified onset or a difference between the time stamp of the identified onset and the time stamp of the predetermined time. The data series recorded by a sensor of the wearable motion system may be a data series recorded by the sensor of the wearable motion system that was exposed to the mechanical perturbation, or any other sensor of the wearable motion system that is synchronised with said sensor.

The start of the recording from the reference motion sensing system can be identified as the start of a data series from the reference motion sensing system. The method may comprise identifying a portion of a data series recorded by the sensor of the wearable motion system that comprises a signal corresponding to the mechanical perturbation. For example, identifying a portion of the data series may comprise identifying a portion of the data series that comprises one or more peaks above a first predetermined threshold and optionally below a second predetermined threshold in the data from the sensor (e.g. accelerometer data). The portion of the data series may start at or at a predetermined time before a detected onset of the first peak detected above a first predetermined threshold and optionally below a second predetermined threshold. The portion of the data series may finish at or at a predetermined time after last peak detected above a first predetermined threshold and optionally below a second predetermined threshold.

Identifying a portion of a data series recorded by the sensor of the wearable motion system that comprises a signal corresponding to the mechanical perturbation may comprise searching a data series recorded by the sensor of the wearable motion system for a signal that satisfies one or more criteria (such as e.g. a signal above a predetermined magnitude). The searching may be performed within a predetermined window of time. Detecting the onset of the signal corresponding to the mechanical perturbation in the data recorded by the sensor may comprise detecting the earliest time stamp at which a data series recorded by the sensor (or a portion thereof) is above a predetermined threshold, and identifying the first local minimum that precedes said time stamp as the onset of the signal corresponding to the mechanical perturbation in the data recorded by the sensor.

The method may further comprise decoding metadata encoded in the data recorded by a sensor of the wearable motion sensing system using a signal in the data corresponding to a mechanical perturbation to which the sensor was exposed. The mechanical perturbation may be the same mechanical perturbation used to synchronise the sensor of the wearable motion system with the reference motion sensing system according to the above embodiments. In other words, the mechanical perturbation that is associated with a predetermined time in the reference motion sensing system may also be configured to encode metadata in the data recorded by the sensor of the wearable motion sensing device. The mechanical perturbation may be configured to generate a signal in the data of the sensor that is exposed to the mechanical perturbation, the signal having a predetermined pattern that can be detected and associated with corresponding metadata. The method may comprise detecting a predetermined pattern in the data recorded by the sensor and identifying metadata associated with the predetermined pattern.

The method may comprise exposing the sensor of the wearable motion system to the mechanical perturbation (optionally at a predetermined time, which may be the start of the recording from the reference motion sensing system). The method may comprise identifying a portion of a data series recorded by the sensor of the wearable motion system that comprises a signal corresponding to the mechanical perturbation. For example, identifying a portion of the data series may comprise identifying a portion of the data series that comprises one or more peaks above a first predetermined threshold and optionally below a second predetermined threshold in the data from the sensor (e.g. accelerometer data). The portion of the data series may start at or at a predetermined time before a detected onset of the first peak detected above a first predetermined threshold and optionally below a second predetermined threshold. The portion of the data series may finish at or at a predetermined time after last peak detected above a first predetermined threshold and optionally below a second predetermined threshold. The method may comprise detecting the onset of the signal corresponding to the mechanical perturbation in the data recorded by the sensor.

The predetermined pattern may comprise one or more peaks that satisfy one or more predetermined criteria. The method may comprises determining whether a peak that satisfies the one or more predetermined criteria is present at each of a plurality of time points. The presence of a peak that satisfies the one or more predetermined criteria at each of the plurality of time points may be interpreted as a first value of a binary representation and the absence of a peak that satisfies the one or more predetermined criteria is interpreted as a second value of the binary representation. The plurality of time points may be a plurality of integer multiples of a predetermined time period from a detected onset of the predetermined pattern. The one or more predetermined criteria may be criteria applying to the height and/or shapes of the peaks and/or the distances between a plurality of peaks. For example, a predetermined criterion may be selected from: the peak having a height above a first predetermined value, the peak being a peak with a single local maximum, the peak being a peak without a shoulder, the peak being a local maximum with a height above a predetermined value, the peak being separated from the nearest peak by a distance above a predetermined value, etc. The method may comprise obtaining the metadata by searching a database or memory storing metadata in association with a respective predetermined pattern in the signal of the sensor and/or a binary code derived therefrom.

The methods of the present aspect can comprise the steps of any embodiment of any other aspect described herein, such as e.g. the first, second, or third aspects described above.

According to a fifth aspect, there is provided a computer-implemented method of analysing motion sensing data, the method comprising: performing the method of any embodiment of any preceding aspect (such as e.g. any embodiment of the first, second, third or fourth aspects), and comparing the synchronised data from the wearable motion sensing device and the reference motion sensing system to identify one or more motion features and/or clinically relevant parameters derived from the wearable motion sensor that are validated in the reference motion sensing system. The method may further comprise obtaining motion sensing data associated with a subject from a wearable motion sensing system; and determining the value of one or more validated motion features and/or clinically relevant parameters derived from the motion sensing data from the wearable motion sensing system. The one or more validated motion features may comprise a gait feature. The motion sensing data associated with a subject may be recorded while the subject is performing one or more tests, such as e.g. static balance tests or active tests. The one or more validated motion features may be biomarker values. A motion feature may be a gait feature. Biomarker values may be values of one or more motion features derived from motion sensing data that are known to correlate with a clinically relevant parameter.

According to any embodiment of any aspect, the method may further comprise providing to a user, through a user interface, one or more of the value of a time lag identified, a first signal, a second signal, synchronised data from the wearable motion sensing system and/or the reference motion sensing system, decoded metadata encoded in the data recorded by a sensor, one or more motion features, one or more clinically relevant parameters derived from the motion sensing data, and a report comprising any one or more of the above.

According to a further aspect, there is provided a system comprising: a processor; and a computer readable medium comprising instructions that, when executed by the processor, cause the processor to perform the (computer-implemented) steps of the method of any preceding aspect (e.g. any of the first, second, third, fourth or fifth aspects). The system may further comprise one or more of: one or more wearable motion sensors, one or more motion marker tracking systems, one or more ground reaction force sensor systems, and one or more mechanical perturbation means.

According to a further aspect, there is provided a non-transitory computer readable medium or media comprising instructions that, when executed by at least one processor, cause the at least one processor to perform the method of any embodiment of any of the first, second, third, fourth or fifth aspects.

According to a further aspect, there is provided a computer program comprising code which, when the code is executed on a computer, causes the computer to perform the method of any embodiment of any of the first, second, third, fourth or fifth aspects.

### BRIEF DESCRIPTION OF THE FIGURES

FIG. 1 shows an embodiment of a system for analysing motion features, such as gait features, of a subject, according to the disclosure.

FIG. 2 is a flow diagram showing, in schematic form, a method of analysing data from motion sensing systems, according to the disclosure.

FIG. 3 is a flow diagram showing, in schematic form, a method of synchronising data from a wearable motion sensor and from a ground reaction force sensing system, according to the disclosure.

FIG. 4 is a flow diagram showing, in schematic form, a method of synchronising data from a wearable motion sensor and from a motion marker tracking system, according to the disclosure.

FIG. 5 is a flow diagram showing, in schematic form, a method of synchronising data from a wearable motion sensor and from a motion marker tracking system, according to the disclosure.

FIG. 6 is a flow diagram showing, in schematic form, a method of analysing motion sensing date from a subject, according to the disclosure.

FIGS. 7A-7D illustrates a method and system for synchronising and/or encoding and decoding information in motion sensing data. FIG. 7A. Mechanical solution to automatically encode and decode information on test type and/or synchronise motion sensing data between one or more wearable motion sensors and a reference motion sensing system. The illustrated system comprises a start button, a computer controlling the reference motion sensing system, and a vibrating motor, which is attached to a wearable motion sensing device (referred to in the examples below as the “master smartphone”) that is internally synchronized with the one or more further wearable motion sensors. Upon an operator pressing the start button, the computer causes a signal to be sent to the vibrating motor, as well as causing the reference motion sensing system to start recording motion data. The signal can be detected in the data from the master smartphone, and used to synchronise the data from the wearable motion sensors (which are internally synchronised) and the reference motion sensing system (which is triggered to start recording at the time of the vibration). The vibrations can additionally be used to encode information, such as e.g. metadata about the recording. This can be used to automatically identify and/or verify metadata that may be otherwise recorded by an operator, for example using the computer controlling the reference motion sensing system. FIG. 7B. The information on the test type can be encoded in a binary vibration pulse sequence, and decoded by detecting the peaks in the acceleration signal during this vibration sequence. Horizontal dashed line indicates the thresholds in the accelerometer magnitude used to identify the vibration peaks. The peaks correspond to ‘1s’ in a binary vibration pulse sequence. Vertical dotted lines indicate the time points at which either a ‘1’ (i.e., a peak in the accelerometer signal), or ‘0’ are expected. Acc, acceleration. FIG. 7C. Example accelerometer data from a master smartphone, showing that the vibrations are localised around the timer start timestamp (vertical dashed line) in the accelerometer signal of the master smartphone. The localization of the peaks is used to identify a binary signal that is decoded to obtain a test type. This can be used to annotate the data, and to validate a test type recorded by an operator. FIG. 7D. Example accelerometer data from a master smartphone, showing that the first peak (right vertical dashed line) is localised in the vibration train in the accelerometer data from the master smartphone. The accelerometer data from the master smartphone is used to detect the onset of the first peak (indicating the start of the recording from the reference motion sensing system), indicated by the left vertical dashed line. The onset detected is used to calculate an offset (time lag) between the wearable motion sensors (including the master smartphone, internally synchronised) and the reference motion sensing system.

FIG. 8 shows an example of a vertical acceleration measured with a smartphone and estimated for a reflective motion capture (Vicon) marker during a 2MWT after initial synchronization with the vibration-based method. Acc, acceleration. The data shows that the synchronization was mostly successful but that in this specific embodiment a small lag remains observable, likely due to a difference in the exact timing at which the reference motion sensing system starts recording and the vibrating motor starts vibrating, upon an operator triggering both actions (e.g. by pressing the start button on FIG. 7A).

FIGS. 9A-9D illustrates schematically an example of a gravity-based method for motion data synchronization (FIG. 9A), an acceleration-based method for motion data synchronization (FIG. 9B), and a force-based synchronization (FIG. 9C). FIG. 9A. The illustrated gravity-based method involves two transformation steps: the first transformation is a transformation of the unit gravity direction measured in M to L, where M is the motion capture reference frame (gravity pointing downwards, aligned to z axis) and L is the local smartphone reference frame (defined using markers on the smartphone, see below) using rotation matrix R_{LM} (step 1), estimate rotation matrix R_{GL} that aligns one of the smartphone local reference frame axis with gravity (step 2); the second transformation transforms the unit gravity direction measured in G to L using the matrix R_{GL} (step 3). Once transformed, the unit gravity direction estimated in steps 1 and 3 can be compared with each other through cross-correlation. FIG. 9B. The illustrated acceleration-based method involves the following steps: 1. the position of a marker on the smartphone measured by the motion capture system in reference frame M is differentiated to obtain a marker acceleration, thereby obtaining a calculated marker acceleration in reference frame M. 2. The three markers on the smartphone are used to transform the smartphone acceleration from reference frame L reference frame M. 3. The contribution of gravity is separated from the smartphone acceleration measurements, thereby obtaining a gravity corrected smartphone acceleration in reference frame M. 4. The two accelerations (the gravity corrected smartphone acceleration from step 3 and the calculated marker acceleration from step 1, both in reference frame M) are compared through cross-correlation to determine the lag. FIG. 9C. CoM acceleration-reaction force synchronization (also referred to as “force-based method). The diagram schematically illustrates the principle behind the method. FIG. 9D. This shows an example of a calculated and measured force signal before and after cross-correlation based alignment using the method illustrated by reference to FIG. 9C.

FIGS. 10A-10C shows distributions of the lags between smartphone and Vicon motion-capture system for different test types as estimated through cross-correlation using (FIG. 10A) acceleration-based lag estimation method, (FIG. 10C) force-based lag estimation method or (FIG. 10B) gravity-based lag estimation method. 2MWT, Two-Minute Walk Test; SBT, Static Balance Test; UTT, U-Turn Test. The lags shown are post-correction lags. Post-correction lags are similar across all active tests for all methods.

FIG. 11 shows results of motion sensing data synchronization with an acceleration-based method. (A,B) Example of a cross-correlation between smartphone's (acc.y) and Vicon's marker's (mdd.y) acceleration before (A) and after (B) applying an acceleration-based synchronization. (C) Scatter plot illustrating the correlation between the smartphone's acceleration (vertical axis) and that of the reflective Vicon markers (horizontal axis) after post-synchronization correction with the force-based method for gait tests.

FIG. 12 shows results of motion sensing data synchronization with a gravity-based method. Y-axis of the reoriented sensor data of the smartphone and the motion capture system for a single 2MWT (fast-paced condition) before (top panel) and after (bottom panel) applying a gravity-based synchronization correction.

FIG. 13 illustrates schematically a clinical study protocol used to obtain data in examples of the disclosure. The protocol comprises an on-site visit at a gait lab (labelled at session 2) where motion sensing data is recorded from a plurality of subjects using both wearable motion sensors (e.g. a plurality of smartphones equipped with the FloodLight system; one or more GaitUp sensors) and a reference motion sensing system. The motion sensing data is recorded while subjects are performed a predetermined series of tests including active tests (e.g. 2 minutes walking test, U-turn test, static balance tests). This data from both types of sensors is synchronised using methods as described herein, and the information derived from this is used to analyse and/or validate the motion sensing data obtained from wearable motion sensors outside of the clinical setting (indicated as “remote” period). 2MWT, Two-Minute Walk Test; 9HPT, 9-Hole Peg Test; ABC, Activities-specific Balance Confidence scale; CVLT-3, California Verbal Learning Test, Third Edition; DaS, Draw a Shape; FSMC, Fatigue Scale for Motor and Cognitive functions; IPS, Information Processing Speed Test; MS, multiple sclerosis; MSIS-29; 29-item Multiple Sclerosis Impact Scale; MSIS-29vs2, 29-item Multiple

Sclerosis Impact Scale version 2; MSSS-88, 88-item Multiple Sclerosis Spasticity Scale; MSWS-12, 12-item Multiple Sclerosis Walking Scale; MSWS-41, 41-item Multiple Sclerosis Walking Scale; PT, Pinching Test; SBT, Static Balance Test; SDMT, Symbol Digit Modalities Test; T25FW, Timed 25-Foot Walk; UTT, U-Turn Test.

FIG. 14 shows schematically the process for jointly analysing data from multiple types of motion sensors used in examples of the disclosure. As illustrated, a plurality of smartphones (top 3 lines, comprising smartphones worn by a subject—top line, a smartphone used as a master smartphone—second line from the top, and a smartphone used as a timer smartphone—third line from the top) are synchronised with each other and with a GaitUp master sensor by mechanical perturbation (illustrated as “first mechanical sync”). The GaitUp master sensor is internally synchronised with any other GaitUp sensor through processes outside of the scope of the present disclosure. However, including the GaitUp sensors in the synchronization by mechanical perturbation ensures that it is also possible to internally synchronise the GaitUp sensors using the mechanical perturbation, for example when the build-in GaitUp synchronization fails. When an operator starts the recording of the reference motion sensing system (indicated as “Vicon”), this triggers vibration of a motor physically coupled to the master phone, and optionally also to the master GaitUp sensor. This is detected in the signal of the master smartphone (indicated as “vicon vibration signal”) and is expected to coincide with the Vicon recording window illustrated as the outer shaded region (Vicon recording window). The operator then records a timestamp for start of the test (indicated as “gait active test begin”), and end of the test (indicated as “gait active test end”). The test window is the window of time between these timestamps and is indicated as the inner shaded area. Multiple such recordings may be obtained (one for each test). At the end of a session, the plurality of smartphones may be synchronised again with each other and with a GaitUp master sensor and other GaitUp devices by mechanical perturbation (illustrated as “second mechanical sync”). This may be used to correct for drift in the clocks of the sensors. Indeed, even if two devices are synchronized, their clocks may have different drifts which may result in different lags being present after a period of time since the first synchronisation.

FIGS. 15A-15C shows results of wearable motion sensor synchronisation by mechanical perturbation. FIG. 15A, FIG. 15B. Internal smartphone synchronization: A. gyroscope data for the master smartphone, showing the characteristic pattern associated with the first and second mechanical synchronizations (before and after testing session). B. Alignment of gyroscope signal from master smartphone with timer smartphone using first mechanical perturbation (left), showing the lag between smartphones at the second mechanical perturbation (right). The same process is repeated for each smartphone with respect to a reference smartphone (here chosen as the master smartphone by convention, although any other smartphone could be used). FIG. 15C, FIG. 15D. Synchronization between smartphones and GaitUp sensors. The GaitUp sensors are synchronized in a similar manner, together with the smartphones. The gyroscope data shows a similar pattern of oscillation as the smartphone data (not shown), which can be used to synchronize the GaitUp sensors with the smartphones. Thus, these figures show that the mechanical perturbation (vibration based) method described herein is able to align time series and correct for drifts between two different types of motion sensors. C. GaitUp devices drift compare to smartphone shows that the GaitUp sensors although well synchronized with each other have a different drift compared to the smartphone sensors. D. Comparison of GaitUp sensor data and smartphone sensor data post synchronization correction using mechanical perturbation. This shows data for the beginning of an active test, showing that the smartphone and GaitUp sensors have been properly synchronised.

FIG. 16 shows comparisons of distributions of differences between heel strike events between GaitUp sensors and motion capture system recordings when using different synchronization methods. In this data, correct synchronization between the smartphones and the motion capture system should result in differences between the mocap located events and the GaitUp located events being close to 0 since the GaitUp synchronization with the mocap depends on the smartphone synchronization with the mocap through the mechanical perturbation based joint synchronization of the smartphones and GaitUp sensors. The left plot compares the distribution of differences between localized heel strike events in the GaitUp vs motion capture system when using only the vibration-based method described in the examples (FIG. 7 and Example 1) to synchronize the data between the GaitUp sensors and the motion capture system (left distribution, in orange), with the distribution of differences when using the gravity based method and the acceleration-based method (data for some active tests synchronized with the former and data for other active tests synchronized with the latter) described in the examples (right distribution, blue). The latter distribution is centred much closer to 0 than the former distribution (vibration only), showing that the use of the gravity-based method and the acceleration-based method alone is suitable to synchronize the data, and performs better than the vibration method alone in this particular set up. The middle plot compares the gravity and acceleration-based methods (distribution in blue with the shoulder on the left) and the force-based method described in the examples (green distribution). This shows that all methods perform well (with averages close to 0), but that the force-based method is more robust (fewer cases that have differences far away from 0). The right plot compares the vibration-based method alone (left distribution in orange) and the force-based method alone (right distribution in green). The data shows that the force-based method can be used alone to generate good, robust synchronization results. HS-heel strike events.

FIG. 17 shows data illustrating the problem of calculating derivatives in the presence of noise. The data shows a signal (left) and its derivative (right), with simulated noise (oscillating orange curves, yn), compared to the true (noise free, theoretical) signal (V/step shaped blue curves, y). Axes are arbitrary units. The plots also show the results of applying a generalised cross-validation (GCV) spline method to the signal in orange (Syn—smooth curves following the V and step shaped true signals). A GCV spline can be used to smooth the signal itself (see curve ys, green, on left plot) and or the first and/or second derivatives. Any differentiation method that is robust to noise may be used in the context of the present disclosure, including e.g. a smoothing algorithm such as a Kalman filter.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The methods described herein focus on the combined analysis (including synchronisation) of data from a plurality of motion sensing devices or systems of different types. In particular, the methods described herein relate to the combined analysis of data from (a) one or more wearable motion sensors, and (b) one or more reference motion sensing systems.

The term “motion sensing device” refers to a device or system comprising one or more sensors configured to record information associated with the movement of a subject. Motion sensing devices used in the present disclosure can be of different “types”. Different types of motion sensing devices can rely on different technologies (e.g. different types of sensors). A first type of motion sensing device may be a wearable motion sensing device. In the context of the present disclosure, a wearable motion sensing device is typically an inertial measurement unit (IMU) and therefore the terms “wearable motion sensor” and “IMU” are used interchangeably. A second type of motion sensing device may be a motion marker tracking system, which captures kinematics by recording the location of one or more markers. A third type of motion sensing device may be a ground reactive force sensing system or “GRF system”. A motion capture system of “mocap” may comprise one or both of: a motion tracking system and a GRF sensing system. In the context of the present disclosure, motion sensing devices or systems that are not IMUs are used as “reference” motion sensing devices/systems. Their data can be analysed together with that from wearable motion sensors, for example to validate the use of the latter for a particular purpose (e.g. to quantify a particular metric of interest such as a motion feature, gait feature, biomarker, clinically relevant parameter, etc.). A reference motion sensing device/system may be a motion capture system. A motion sensing device typically records a time dependent signal, at a predetermined sampling frequency (such as e.g. 50 Hz, 100 Hz, etc.) The predetermined sampling frequency may be dependent on the motion sensing device. In any method described herein, comparison or any other joint analysis of signals from a plurality of motion sensing devices that have different sampling frequencies may comprise downsampling or upsampling the data from one or more of the motion sensing devices to obtain data with the same sampling frequency as another one of the one or more motion sensing devices. Downsampling may be used in any embodiment described herein to obtain data with the same sampling frequency from a plurality of motion sensing devices. Upsampling may be used in any embodiment described herein to obtain data with the same sampling frequency from a plurality of motion sensing devices. Upsampling may be performed by interpolating a data series. A combination of downsampling and upsampling may be used, such as e.g. downsampling the data from one motion sensing device and upsampling the data from another motion sensing device. In embodiments, upsampling can improve the temporal resolution of a signal. For example, upsampling may be performed in the frequency domain (e.g. interpolating a time series by zero padding of a corresponding frequency domain signal obtained using a direct Fourier transform, preferably by adding zeros after the first N/2 spectral samples where N is the length of the original time series, then taking the inverse direct Fourier transform of the padded signal) through padding and then the data may be converted back to the time domain (see e.g. Lyons 2004).

A “wearable motion sensor” as used herein refers to an inertial measurement unit (IMU) that can be worn by a subject. The words “wearable motion sensor” and “IMU” are used herein interchangeably. A device that can be worn by a subject is a device that is portable. The device may not necessarily be integrated into a garment or wearable accessory (e.g. band, watch, bag, etc.), although it may be integrated into a garment or wearable accessory. An IMU is a device that measures linear acceleration and rotational rate (angular velocity). IMUs typically comprise a gyroscope and an accelerometer. A gyroscope measures angular velocity of the sensor. An accelerometer measures acceleration of the device and gravitational acceleration.

IMUs can further comprise a magnetometer, although this is not necessary in the methods described herein. In practice, IMU functionalities are frequently included in portable devices such a smartphones. Thus, reference to a wearable motion sensor may refer to a smartphone that is equipped with an accelerometer and gyroscope. Other types of IMUs are however possible and envisaged. For example, dedicated devices for inertial measurement exist, optionally with additional functionalities related to specific purposes, such as e.g. the GaitUp sensor (also referred to as “Physilog™”). Any portable IMU may be used as a wearable motion sensor in the context of the present disclosure. The methods described herein may use one or a plurality of wearable motion sensors. A “reference motion sensing system” is any motion sensing system that is not a wearable motion sensing device (IMU) as described herein. As described herein, a reference motion sensing system can be or comprise a marker tracking system or a ground reaction force sensing system (or a system comprising both a marker tracking system and a ground reaction force sensing system).

A motion marker tracking system (also referred to herein as “marker tracking system” or “marker tracking sensor”) is a system that is configured to record the movement of a subject by detecting the position of a plurality of markers associated with the subject. For example, a motion marker tracking system may comprise a plurality of cameras that record the position in 3 dimensions of a plurality of markers. The plurality of markers may be reflective markers and the plurality of cameras may be infrared cameras. The plurality of markers may be located on the subject and/or on wearable motion sensors worn by the subject. Markers located on the subject (such as e.g. on clothing) can be located at predetermined or arbitrary locations. Advantageously, at least one marker can be located on a wearable motion sensor. In embodiments, at least 3 markers that are not collinear are located on a wearable motion sensor. This enables to establish a system of coordinates associated with the wearable motion sensor, in the system of coordinates of the motion capture system (in this case comprising a marker tracking sensor), as will be described further herein.

A ground reaction force (GRF) sensing system is a system comprising one or more ground reaction force sensors. GRF sensors are known in the art and can be selected from: force plates (e.g. triaxial force plates), instrumented treadmill devices (e.g. treadmills comprising one or more force plates), instrumented stairs or ground plates (e.g. stairs or floor plates comprising one or more force plates) and wearable force sensors attached to or integrated in shoes (e.g. specially adapted shoes comprising one or more force sensors). A ground reaction force sensor is a sensor that measures the force exerted by the ground on the subject's body as the subject's body contacts the ground. A ground reaction force sensor can also be seen as a sensor that measures the force exerted by the subject's body on the sensor, where the sensor is associated with a contact point between the subject and the ground. Any sensor that measures the direction of forces applied on a foot or feet of the subject may be used. Some GRF sensing systems may also measure torque (e.g. force plates measure forces and moments exerted by the subject's body on the sensor). This is not necessary for use in the methods described herein. The magnitude of the forces is also not necessary for use in the methods described herein (the direction/vector magnitude is sufficient). The GRF of a subject varies while the subject is walking, running, or jumping due to acceleration forces associated with the subject's movement.

The methods herein may make use of cross-correlations to align two signals. Cross-correlation is a measure of similarity of two time series as a function of the displacement of one relative to the other. This may also be referred to as a convolution operator, sliding dot product or sliding inner product. This may be used to identify a displacement or lag that, when applied to one of the two signals, maximises the similarity with the other of the two signals.

Various embodiments of the methods described herein determine a rotation matrix that aligns a first reference frame (also referred to as a coordinate system) with a second reference frame. Such matrices may be used to transform a vector in the first reference frame into a corresponding vector in the second reference frame. Such a matrix may be calculated as a direction cosine matrix (DCM). A DCM contains as coefficients the dot products of every pair of unit vectors defining the two orthogonal vector bases (e.g. a point with scalar components [p,q,r] in basis [L1,L2,L3] can be transformed into a point with scalar components [u,v,w] in basis M defined by vectors [M1,M2,M3] by multiplying the vector [p,q,r] by a 3×3 matrix C with coefficients [c11=M1.L1, c12=M1.L2, etc.] (where “.” refers to the dot product). The same principles can be used to obtain any rotation matrix between two orthogonal vector bases in the methods described herein.

The systems and methods described herein may be implemented in a computer system, in addition to the structural components and user interactions described. As used herein, the term “computer system” includes the hardware, software and data storage devices for embodying a system or carrying out a method according to the above-described embodiments. For example, a computer system may comprise one or more central processing units (CPU) and/or graphics processing units (GPU), input means, output means and data storage, which may be embodied as one or more connected computing devices. Preferably the computer system has a display or comprises a computing device that has a display to provide a visual output display. The data storage may comprise RAM, disk drives, solid-state disks or other computer readable media. The computer system may include a plurality of computing devices connected by a network and able to communicate with each other over that network. It is explicitly envisaged that computer system may consist of or comprise a cloud computer.

The methods described herein are computer implemented unless context indicates otherwise. Indeed, the amount of data involved in analysis of wearable motion sensor data is such that the methods described herein are far beyond the capability of the human brain and cannot be performed as a mental act. For example, data from a single wearable motion sensor can comprise values for at least acceleration along 3 axes recorded at high sampling frequencies such as e.g. 100 Hz leads to 300 data points per second, recorded over dozens or hundreds of seconds in realistic clinically relevant testing scenarios, leading to at least 18,000 data points per minute for the accelerometer data alone from a single sensor, to be processed (including e.g. cross-correlation, numerical differentiation, etc.) and compared to data from motion capture systems comprising marker tracking systems and/or ground reaction force systems comprising similar amounts of data per sensor, where such systems typically include a plurality of sensors. Such operations simply cannot performed mentally.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The disclosure relates to analysis of motion sensing data comprising both data from one or more wearable motion sensors (also referred to as inertial measurement units, IMUs) and one or more reference motion sensing systems including marker tracking systems and/or or ground force sensing systems. The disclosure provides methods that enable the combined analysis of such data, by synchronising the two types of data with each other. As described herein, the motion sensing data from a plurality of wearable motion sensors may also be synchronised through mechanical perturbations. As described herein, the data from a plurality of wearable motion sensors may be automatically annotated with metadata integrated in the data, and/or synchronised with a reference motion sensing system through mechanical perturbation triggered by a control device coupled to the reference motion sensing system.

### Systems

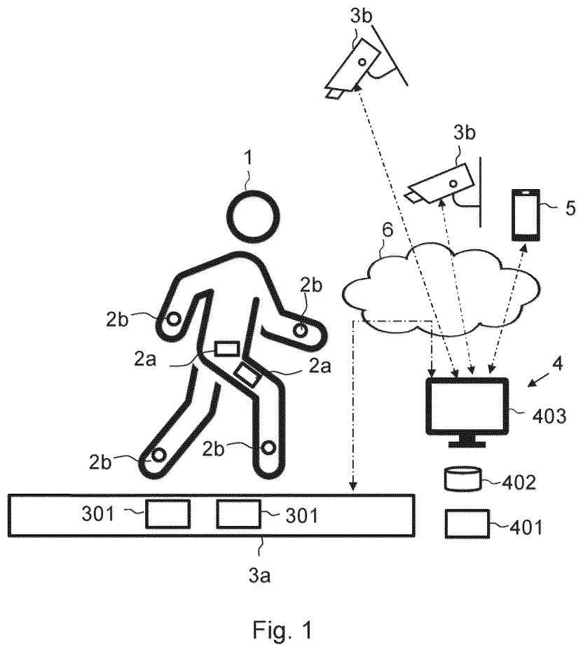

FIG. 1 shows an embodiment of a system for analysing motion sensing data from a subject 1, for characterising a subject 1 (e.g. determining whether a subject has a neurological dysfunction, is likely to respond positively or negatively to a course of treatment, etc.) and/or for providing a treatment recommendation for a subject 1 and/or for selecting a subject 1 for participating in a clinical trial, and/or for monitoring a subject, according to the present disclosure. The system comprises a computing device 4, which comprises a processor 401 and computer readable memory 402. In the embodiment shown, the computing device 1 also comprises a user interface 403, which is illustrated as a screen but may include any other means of conveying information to a user such as e.g. through audible or visual signals, by producing a report, etc. The computing device 4 is communicably connected, such as e.g. through a network 6, to one or more reference motion sensing systems. In the illustrated embodiment, the reference motion sensing system comprises a motion capture system comprising a plurality of cameras 3b (e.g. infrared cameras) which together form a marker tracking system and a ground reaction force sensing system 3a comprising a support (e.g. a treadmill, ground or stairs) with a plurality of ground reaction force sensors 301. The subject 1 is wearing a plurality of wearable motion sensors 2a, 2b. The wearable motion sensors 2a, 2b may comprise a first type of wearable motion sensors 2a (e.g. one or more smartphones), and a second type of wearable motion sensors 2b (e.g. one or more GaitUp sensors). Any inertial measurement unit (IMU) may be used as sensors 2a, 2b. The wearable motion sensor(s) 2a and/or 2b may be worn by the subject at predetermined locations. Alternatively, the sensors 2a and/or 2b may be worn by the subject at arbitrary locations. At least one wearable motion sensor may advantageously be worn by the subject near the subject's centre of mass, such as e.g. in their waist region. For example, at least one of the sensors may be attached or included in a belt of waist bag, or otherwise integrated in a garment around the waist of the subject. As shown, the system further comprises a further IMU 5 that comprises a mechanical perturbation unit (e.g. a vibrating motor) communicably coupled to the computing device 4. The computing device may be a smartphone, tablet, personal computer or other computing device. The computing device is configured to implement a method for analysing motion sensing data, as described herein. In alternative embodiments, the computing device 4 is configured to communicate with a remote computing device (not shown), which is itself configured to implement a method as described herein. In such cases, the remote computing device may also be configured to send the result of the method to the computing device. Communication between the computing device 1 and the remote computing device may be through a wired or wireless connection, and may occur over a local or public network 6 such as e.g. over the public internet.

### Methods of Analysing Motion Sensing Data

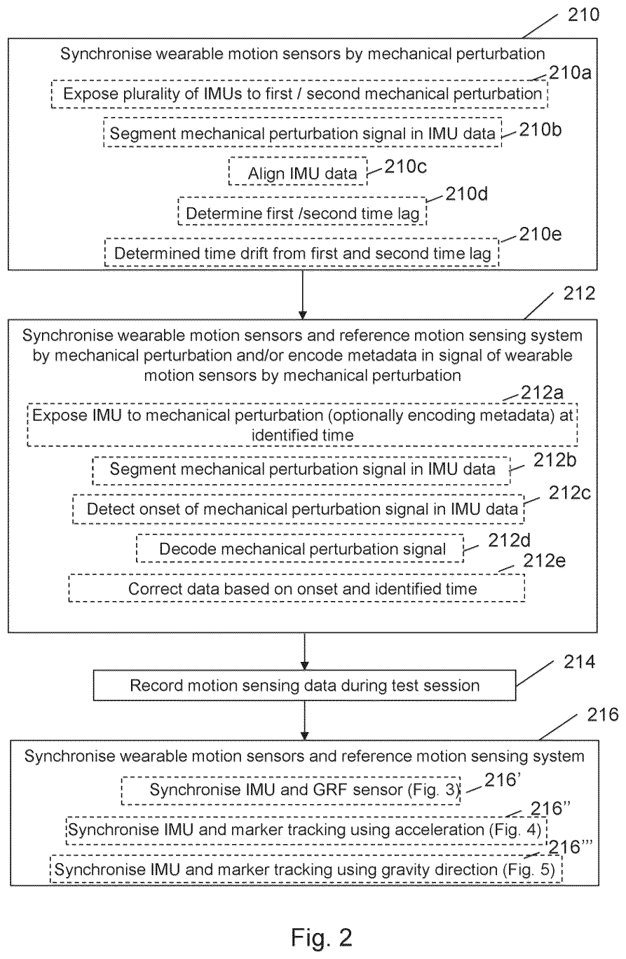

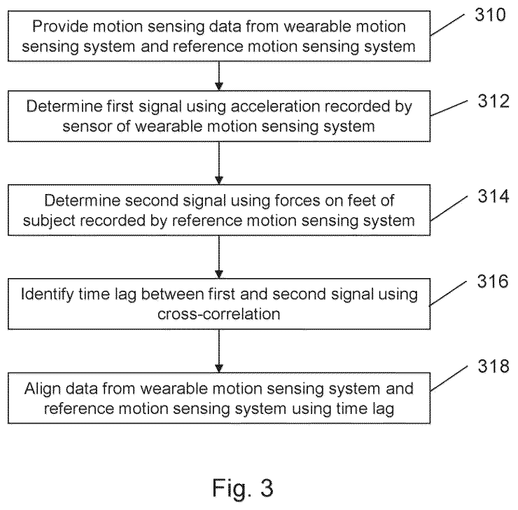

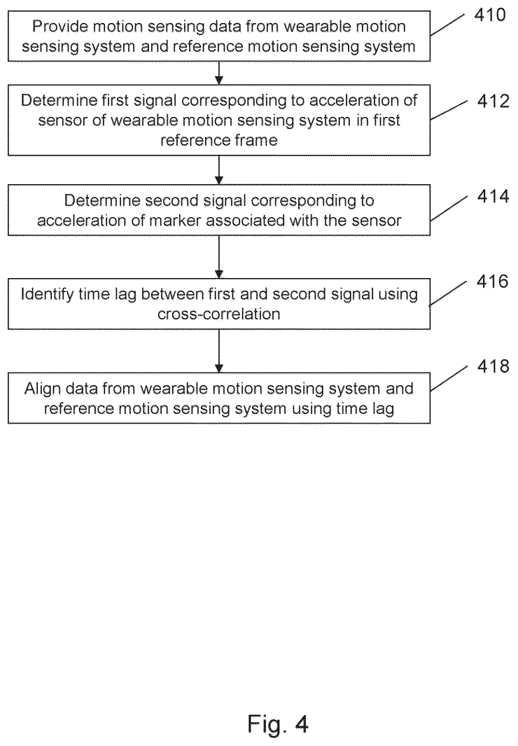

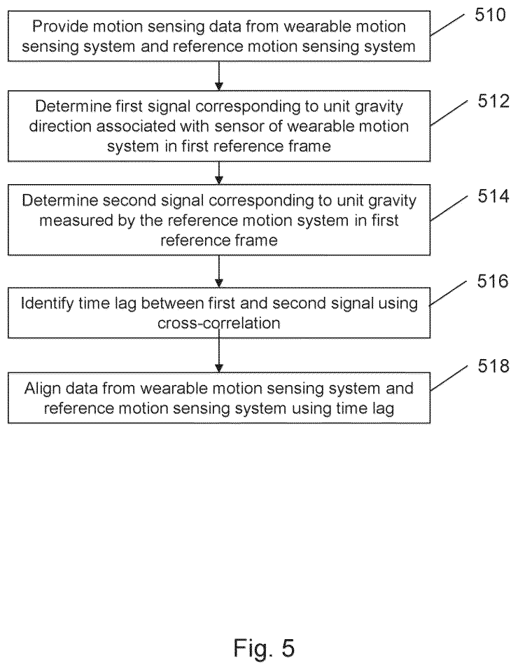

The present disclosure provides methods of analysing motion sensing data from multiple different motion sensing systems including at least one wearable motion sensor. The methods may comprise one or more steps selected from: synchronising data from one or more wearable motion sensors and one or more GRF systems (as will be explained in more detail by reference to FIG. 3), synchronising data from one or more wearable motion sensors and a marker tracking system based on acceleration signals (as will be explained in more detail by reference to FIG. 4), synchronising data from one or more wearable motion sensors and a marker tracking system based on the detected gravity direction (as will be explained in more detail by reference to FIG. 5), synchronising a plurality of wearable motion sensors by mechanical perturbation (as will be explained in more detail by reference to FIG. 2), synchronising data from a system comprising a wearable motion sensor pre-synchronised with the one or more wearable motion sensors, and a reference motion sensing system (such as e.g. a GRF system and/or marker tracking system) that is not pre-synchronised with the wearable motion sensors (as will be explained in more detail by reference to FIG. 2), and encoding metadata about motion sensing data by mechanical perturbation of a communication device associated with one or more wearable motion sensors (as will be explained by reference to FIG. 2). Any one or more (or even all) of the above steps may be performed. For example, FIG. 2 illustrates an embodiment in which a plurality of the above steps are performed as part of a method of analysing motion sensing data.

FIG. 2 is a flow diagram showing, in schematic form, a method of analysing motion sensing data. At step 210, a plurality of wearable motion sensors (abbreviated as “IMU”, in FIG. 2) are synchronised by exposing the plurality of sensors to a mechanical perturbation (step 210a). Any mechanical perturbation that is detectable in the wearable motion sensor data may be used. In particular, any mechanical perturbation that can be detected by an accelerometer and/or a gyroscope may be used. Advantageously, the mechanical perturbation is selected such that the signal produced in accelerometer and/or a gyroscope data is distinguishable from other signals that may be recorded in normal use of the wearable motion sensors. For example, the mechanical perturbation may comprise an oscillation around an axis (i.e. a periodic rotational movement around an axis, covering a predetermined range or rotational angles). Such a movement can be very easily detected in gyroscope data. Synchronising the sensors by mechanical perturbation can comprise simultaneously exposing the sensors to a mechanical perturbation, such as e.g. by placing all sensors to be synchronised in a common physical structure and applying the mechanical perturbation to the common physical structure. Synchronising the sensors by mechanical perturbation can further comprise identifying a region of a signal from each of the sensors (e.g. a portion of the gyroscope signal from each sensor) that comprises a signal corresponding to the mechanical perturbation. This may also be referred to as segmenting the sensor data (step 210b). Synchronising the sensors by mechanical perturbation can comprise aligning a signal from each sensor (or a segmented region thereof) by cross-correlation (step 210c). Aligning segmented regions that comprise a signal corresponding to the mechanical perturbation may advantageously be more robust than aligning non-segmented signals. In particular, one of the sensors may be designated as a reference sensor, and cross-correlation may be used to identify a time lag to be applied to each sensor to align the signal from the respective sensor with the reference sensor (step 210d). The time lag can be seen as an interval of time that when used to shift the signal of the sensor, results in the highest similarity between the signals associated with the mechanical perturbation in the sensor and the reference sensor. The reference sensor may be any of the plurality of wearable motion sensors that have been synchronised. Time lags occur because of differences between the internal clocks of the plurality of sensors, resulting in timestamps associated with data from the same physical event being different.

The plurality of wearable motion sensors comprise one or more wearable motion sensors that, in use, are worn by a subject and used to record their movements. Optionally, the plurality of wearable motion sensors can comprise a wearable motion sensor device that is used for synchronisation with a reference motion sensing system and/or encoding of metadata in the signal of the one or more wearable motion sensors (step 212). The latter may be referred to as a “master wearable motion sensor” or “master sensor”. In embodiments, the master device is used as the reference device for the purpose of step 210. Optionally, the one or more wearable motion sensors can comprise a device that is used to record one or more timestamps associated with a recording, such as the start and end time of a recording. This may be referred to as a “timer device”, or “timer sensor”. The timer device (if present), master device (if present) and one or more wearable motion sensors worn by a subject are all synchronised with each other (also referred to as “internally synchronised”) at step 210.

Step 210 may comprise exposing the plurality of sensors to a mechanical perturbation at a first time (such as e.g. prior to recording of the motion sensing data at step 214) and at a second time (such as e.g. after recording of the motion sensing data (step 214)), in order to identify and optionally correct for a time drift in one or more of the wearable motion sensors (step 210e). A time drift refers to a difference in the amount of time recorded to have passed between two events in one sensor compared to another sensor. In other words, time drifts occur for a pair of sensors when the time lag observed between the sensors at a first time point is not the same as the time lag observed between the sensors at a second time point. Yet another way to look at this is that a time drift occurs when two sensors are synchronized at a first time point (e.g. a time point associated with a first mechanical perturbation), and there is a lag between the two sensors at a second, subsequent time point. Time drifts can occur because of a difference in the speed of passing of time recorded by the clock of two sensors. The time lags identified for a sensor at the first and second time points can be used to scale the time axis of the sensor signals, for example linearly. In other words, the time lags at two different time points can be used to calculate the parameters of a monotonic function) that takes as input a timestamp and produces as output a corrected timestamp, such that when the monotonous function is applied to the data from the sensor, the signal for the mechanical perturbation at the first and second time point occurs at the same respective timestamp as those in a reference device (e.g. the master device if all devices are synchronised to the master device). A monotonous function may be linear or non-linear. For example, the time lag at two different time points can be used to calculate the initial value (b) and the rate of change (a) of a linear equation (y=ax+b) to be applied to the data from a sensor such that the signal for the mechanical perturbation at the first and second time point occurs at the same respective timestamp as those in a reference device. A linear monotonic function may be advantageous when the drift between the clock of the sensor and the reference sensor is linear. This may also be referred to as a sensor drifting linearly relative to a reference sensor.

At step 212, a mechanical perturbation is used to (a) synchronise data from a system comprising the master wearable motion sensor (optionally synchronised with the one or more wearable motion sensors at step 210), and one or more reference motion sensing systems that is not pre-synchronised with the wearable motion sensors; and/or (b) encode metadata in the data of the master wearable motion sensor. Step 212, when used to synchronise the wearable motion sensor(s) and the reference motion sensing system(s) may be referred to as “pre-synchronisation” because a further synchronisation step 216 is applied in the illustrated embodiment. Note that in some embodiments a further synchronisation step may not be used, and conversely in some embodiments a pre-synchronisation step may not be used. In other words, according to embodiments of the disclosure, step 212 may be performed instead or in addition to step 216, and in other embodiments step 216 may be performed instead of (i.e. without) step 216. Step 212 can comprise exposing the master sensor to a mechanical perturbation (step 212a). Any of the wearable motion sensors synchronised at step 210 may be used for this purpose. The use of the master sensor is advantageous because it is not worn by a subject or manipulated by an operator. This simplifies the detection of the signal associated with the mechanical perturbation in step 212. The one or more reference motion sensing systems are typically fixed (i.e. not wearable). The one or more reference motion sensing systems can be a motion capture system. The one or more reference motion sensing systems can include a GRF system and/or a marker tracking system (e.g. a system comprising a plurality of cameras that record the location of a plurality of markers on the subject). When the reference motion sensing system comprises multiple systems, these can advantageously be internally synchronised. For example, they can be associated with the same computing device and as such have timestamps originating from the same internal clock. When step 212 is used to synchronise data between the plurality of wearable motion sensors (optionally internally synchronised at step 210) and the reference motion sensing system(s), the mechanical perturbation is triggered at a time point that is identifiable in the data from the reference. For example, the mechanical perturbation may be triggered at the same time as the reference motion system is triggered to start recording. Therefore, by identifying the time stamp in the wearable motion sensor data associated with the mechanical perturbation and the time stamp in the reference motion sensing system data associated with the start of recording, it is possible to align the data from the two types of sensors. The reference motion sensing system data may record an absolute or relative time (i.e. a time since the start of recording). In such embodiments, the time stamp in the reference motion sensing system data associated with the start of recoding may be 0, and the data from the reference motion sensing system can be aligned to that of the wearable motion sensor by adding the identified timestamp associated with the mechanical perturbation to all timestamps from the reference motion sensing system. In embodiments, the reference motion sensing system records an absolute time, and the data from the two types of sensors may be aligned by calculating a time lag to be applied to the timestamps from the wearable motion sensors (optionally after the timestamps have already been adjusted by synchronisation as explained in relation to step 210) to obtain timestamps corresponding to those of the reference motion sensing system, or vice-versa. The mechanical perturbation can be any mechanical perturbation that is detectable in the signal of a wearable motion sensor, such as e.g. a perturbation resulting in a distinctive pattern in accelerometer and/or gyroscope data. The mechanical perturbation may be one that is detectable using accelerometer data alone. The mechanical perturbation is advantageously one that generates a signal in the wearable motion sensor data that is distinguishable from signals associated with normal use of the wearable motion sensor. The wearable motion sensor that is exposed to the mechanical perturbation can be one that is not worn by a subject, but that has been synchronised (such as e.g. as explained by reference to step 210) with the one or more wearable motion sensors worn by the subject. Use of a wearable motion sensor that is not worn by a subject (i.e. not used to record the subject's motion) but that is synchronised with the wearable motion sensors worn by the subject enables the signal associated with the mechanical perturbation to be simpler to detect. As the skilled person understands, such a wearable motion sensor does not need to be of the same type as those worn by the subject, provided that they can be synchronised with those worn by the subject. Exposing the wearable motion sensor (e.g. master sensor) to a mechanical perturbation can comprise causing a vibration element physically associated with the wearable motion sensor to vibrate. For example, a vibrating motor can be attached to the wearable motion sensor. Alternatively, the wearable motion sensor may already comprise a vibration motor. Other alternatives are possible, such as e.g. positioning the wearable motion sensor in or on a moving support (e.g. a vibrating support). Advantageously, the step(s) of exposing the wearable motion sensor to a mechanical perturbation may be triggered by a process that also triggers the start of recording of the reference motion sensing system(s). For example, a computing device may be configured to both cause the reference motion sensing system(s) to start recording motion data, and to cause a wearable motion sensor to be exposed to a mechanical perturbation (e.g. by communicating with a vibration element associated with the wearable motion sensor).

When step 212 is used to encode metadata in the data of a wearable motion sensor, the pattern of the mechanical perturbation may be configured to encode information, for example using a binary code. For example, the mechanical perturbation may comprise a sequences of individual mechanical perturbations separated in time by integer multiples of a predetermined time interval. The presence or absence of a signal corresponding to the mechanical perturbation in the wearable motion sensor data a plurality of integer multiples of the predetermined time interval can be interpreted as a “1” or “0”. For example, the mechanical perturbations may comprise a sequence of individual perturbations separated by multiples of a time period t, such that the presence of a signal at any of times t0, t0+1*t, t0+2*t, t0+3*t, t0+4*t, etc. can be interpreted as a “1” and the absence of a signal at any of those times can be interpreted as a “0”. As a specific example, the sequence ‘11010111’ can be encoded by a mechanical perturbation comprising a plurality of mechanical perturbations at times t0, t0+1, t0+3, t0+5, t0+6, and t0+7. The time to can be labelled as t=1 and a signal can be interpreted as a “1” or “0” depending on the presence or absence of a mechanical perturbation at each of t=2, 3, 4, 5, 6, 7, etc (where the numbers refer to multiples of the time period t, which can be e.g. seconds). The mechanical perturbation can further comprise a pattern of individual perturbations used to mark the start and end of the mechanical perturbation. For example, a predetermined number of consecutive perturbations separated by a time period t (equivalent to a “11” signal if the predetermined number is “2”) can be used at the start and end of any encoded signal. The t interval used for the start signal may be the same or different to the t interval used for the end signal and/or the t interval used to encode the metadata. The individual mechanical perturbations may be identical. The individual mechanical perturbations may each have a duration that is shorter than the time period t between individual perturbations in a sequence. For example, when the time t is set to 1 s, the individual perturbations may each comprise a specific perturbation lasting 0.3 s (or any a*t where a<1) followed by 0.7 s (or any (1−a)*t) of rest time (time without perturbation). Each individual perturbation may be obtained by actuating the vibrating element associated with the wearable motion sensor. Thus, step 212 may comprise causing a vibration element physically associated with the wearable motion sensor to vibrate. The mechanical perturbation used for encoding metadata can be the same as the mechanical perturbation used to synchronise the wearable motion sensor(s) with the reference motion sensing system.

Step 212 can further comprise identifying a region of a signal from the sensor that has been exposed to the mechanical perturbation (e.g. a portion of the accelerator signal from the master sensor) that comprises a signal corresponding to the mechanical perturbation (step 212b). This may also be referred to as segmenting the sensor data. For example, the data from the sensor that has been exposed to the mechanical perturbation may be searched for a signal that satisfies one or more predetermined criteria. For example, the data may be searched for a signal that is above a first predetermined value and optionally below a second predetermined value. Segmenting the sensor data may make use of information about the identifiable time point in the reference motion sensing system. For example, the data from the sensor that has been exposed to the mechanical perturbation may be searched for a signal that satisfies one or more predetermined criteria comprising a criterion on the distance from the timestamp of the identifiable time point. The timestamp of the identifiable time point may not be the same time stamp as the time stamp at which the signal corresponding to the mechanical perturbation is located (as the wearable motion sensor data has not yet been synchronised with the reference motion sensing system), but it may be expected to be relatively close to it, or at least within a predetermined time period from it.

Step 212 can further comprise identifying the onset of the signal corresponding to the mechanical perturbation in the sensor signal (optionally in the segmented sensor signal) (step 212c). In embodiments, onset detection can be performed by identifying the start of the mechanical perturbation or individual mechanical perturbations comprised in the mechanical perturbation as the first time that the sensor signal (optionally the segmented sensor signal) crosses a predetermined threshold, and detecting a local minimum that precedes this time (e.g. the first value preceding this time where the value is lower than a value that immediately precedes it). Alternatively, onset detection can be performed for example by identifying one or more peaks corresponding to the mechanical perturbation or individual mechanical perturbations comprised in the mechanical perturbation, and identifying the onset of the first identified peak. A peak may be identified as a local maximum of the signal. Any peak detection algorithm known in the art may be used. Any other shape of signal that is expected to be recorded as a result of the mechanical perturbation may be used, where the exact shape of the signal (and the characteristics of the onset of such a signal) may depend on the mechanical perturbation. The time stamp associated with the onset of the signal corresponding to the mechanical perturbation in the wearable motion sensor signal can be used correct the data from the wearable motion sensor signal or the reference motion sensor signal, by using the time stamp associated with the onset of the signal corresponding to the mechanical perturbation in the wearable motion sensor signal as equivalent to the identifiable time point in the reference motion sensing signal (step 212a). For example, as explained above, when both systems record time stamps as absolute times, a time lag between the two systems can be identified by comparing the time stamp associated with onset of the signal corresponding to the mechanical perturbation in the wearable motion sensor signal with the time stamp of the identifiable time point in the reference motion sensor signal. Alternatively, when the reference motion sensing system records a relative time, such as a time relative to the identifiable time (i.e. the identifiable time is the start of the recording from the reference motion sensing system), a time stamp associated with the onset of the signal corresponding to the mechanical perturbation in the wearable motion sensor signal can be added to all timestamps in the reference motion sensor signal. The identified time lag can be used to synchronise the data from the wearable sensor (and any other sensor already synchronised with this sensor) and the reference motion sensing system. Optionally, step 212 may be repeated at a second time point in order to identify a second time lag, from which a time drift between the wearable motion sensor and the reference motion sensing system can be identified. This can be used to align the data from the wearable motion sensor(s) and the reference motion sensing system in a manner similar to that described in relation to step 210.

Step 212 can further comprise decoding metadata encoded in the pattern of the mechanical perturbation, using data from the wearable motion sensor exposed to the mechanical perturbation (step 212d). For example, one or more peaks corresponding to individual mechanical perturbations may be detected (optionally in segmented data from the wearable motion sensor). The one or more peaks may be detected as local maxima that satisfy a plurality of constraints comprising a minimum height constraint and a time between peaks constraint (e.g. a minimum distance between consecutive peaks). For example, one or more peaks may be detected by: identifying a plurality of local maxima as candidate peaks in data or segmented data from the wearable motion sensor, removing local maxima (candidate peaks) that do not satisfy a minimum heigh constraint, and iteratively removing the smallest local maxima (candidate peaks) until all remaining candidate peaks satisfy a minimum distance between peaks constraint. The remaining candidate peaks may be the identified peaks. The location of the detected peaks can be used to obtain a binary representation, such as e.g. by determining whether a peak is observed at each of a plurality of predetermined time intervals from a first detected peak or any other signal identifying the start of a pattern encoding metadata (e.g. a plurality of peaks separated by a predetermined time). For example, a plurality of peaks may be detected, the time location of the peaks may be identified, and whether a peak is detected at each of a plurality of times where peaks are expected to be present may be recorded. For example, presence of a peak at or around any of t0, t0+1*t, t0+2*t, t0+3*t, t0+4*t, etc. may be interpreted as a “1”, and absence of a peak at or around any of t0, tO+1*t, t0+2*t, t0+3*t, t0+4*t, etc. may be interpreted as a “0”. Peaks located within a tolerance from expected times (such as e.g. within 20% or 20% of the value of t from the expected time) may be considered to be located at the nearest expected time. The binary representation may then be converted to metadata using any predetermined conversion scheme. For example, the binary representation may encode letters, numbers, etc. or may simply be associated with a predetermined metadata field stored in a memory. Thus, the metadata itself may be directly encoded, or the encoded data may comprise information from which the metadata can be retrieved (e.g. from a database, lookup table, etc. storing equivalents between encoded data and corresponding metadata). The metadata can comprise information about one or more of: the subject (including e.g. age, sex, clinical parameter, etc.), the operator (e.g. identity, test location), and a particular motion task to be performed during the recoding (e.g. identify of a static balance test or active test to be performed by the subject).

As explained above, in embodiments a method as described herein may comprise implementing step 212, and only optionally steps 214 and 216. This may be advantageous when the onset of the mechanical perturbations and the time point that is identifiable in the data from the reference are expected to correspond well to each other. This may be the case, for example, when the mechanical perturbation are triggered at the same time as the reference motion system is triggered to start recording, and the time between the triggering of the two events and the two events happening is either substantially identical or known.

At step 214, motion sensing data associated with a subject is recorded while the subject is performing one or more tests. The one or more tests may be gait and/or balance tests. The tests may be any kind of guided or non-guide movement. The methods described herein are applicable to the analysis of motion sensing data regardless of the type of motion that is being performed during acquisition of the motion sensing data. The methods described herein have been developed in the context of analysis of motion data for clinical purposes, and may be particularly advantageous in this context since reference motion sensing systems are typically used in such contexts. The data comprises data from the one or more wearable motion sensors and data from the one or more reference motion sensing systems.

At step 216, the data from the one or more wearable motion sensors worn by the subject and from the reference motion sensing system(s) (i.e. data obtained at step 214) is synchronised using only the respective signals from the one or more wearable motion sensors worn by the subject and the reference motion sensing system(s). As explained above, in embodiments of the disclosure step 216 is performed as part of a method that does not comprise step 210 and/or step 212. Thus, also described herein by reference to FIG. 2 are methods that comprise performing steps 214 and 216. The data from each wearable motion sensor worn by the subject may be individually synchronised with the data from a reference motion sensing system. Alternatively, the data from one of the wearable motion sensors may be synchronised with the data from a reference motion sensing system. When a plurality of wearable motion sensors are used, a subset of the wearable motion sensors (such as e.g. one of them) can be synchronised with the reference motion sensing system, and the plurality of wearable motion sensors can be synchronised with each other using methods as described herein (such as e.g. methods of step 210). Step 216 may comprise one or more of: step 216′ of synchronising data from one or more wearable motion sensors (abbreviated as “IMUs” on FIG. 2) and data from one or more GRF sensors comprised in the reference motion sensing system (as will be explained in more detail by reference to FIG. 3), step 216″ of synchronising data from one or more wearable motion sensors and a marker tracking system comprised in the reference motion sensing system based on acceleration signals (as will be explained in more detail by reference to FIG. 4), and step 216″ of synchronising data from one or more wearable motion sensors and a marker tracking system based on the gravity direction (as will be explained in more detail by reference to FIG. 5). When the one or more reference motion sensing systems comprise both a marker tracking system and a GRF system, the data from both can be synchronised with the data from the wearable motion sensors. The data from the marker tracking system can be synchronised with the data from the one or more wearable motion sensors using a method based on acceleration signals and/or using a method based on the gravity direction. In embodiments, the data from the marker tracking system can be synchronised with the data from the one or more wearable motion sensors using a method based on acceleration signals and using a method based on detection of the gravity direction. The results of both methods can be compared to identify the method that results in the highest correlation between the data from the one or more wearable motion sensors and the data from the reference motion sensing system. Similarly, in embodiments where the one or more reference motion sensing systems comprise both a marker tracking system and a GRF system, the data from both can be synchronised with the data from the wearable motion sensors using any one or more of the methods described herein and by reference to FIGS. 3, 4 and 5, and the results of the different methods can be compared to identify the method that results in the best alignment (such as e.g. highest similarity or highest correlation of the signals or metrics derived therefrom such as e.g. gait features) between the data from the one or more wearable motion sensors and the data from the reference motion sensing system. The synchronised data obtained using the identified method may then be used for further analysis.

As the skilled person understands, it is not necessary for the data analysis steps to be performed in the order indicated. For example, the mechanical perturbations of steps 210 and 212 may be executed, the motion sensing data may be recorded at step 214, then the data from the motion sensing devices can be analysed to perform one or more of: synchronising the wearable motion sensors with each other using the mechanical perturbation of step 210, synchronising the wearable motion sensors and the reference motion sensing system using the mechanical perturbation of step 212, and synchronise the wearable motion sensors and the reference motion sensing system as described in relation to step 216. Further, a method as described herein may be performed using data that has been previously acquired using methods as described herein, such as e.g. by reference to FIG. 2. For example, a method as described herein can comprise receiving data from one or more wearable motion sensors that have been exposed to a first (and optionally second) mechanical perturbation, but where the method only comprises the steps of synchronising the data using the signal derived therefrom (e.g. steps 210b, c, d and e, where step a has been previously performed and is not part of the method). Similarly, a method as described herein can comprise receiving data from one or more wearable motion sensors and from one or more reference motion sensing systems, wherein at least one of the wearable motion sensors have been exposed to a mechanical perturbation at an identified time in the data from the reference motion sensing systems, and synchronising these data using steps 212b, c, d, and e. Alternatively, the steps of exposing the respective sensors to mechanical perturbations can be performed as part of the methods described herein. Any mechanical perturbation can be produced by one or more mechanical perturbation means. For example, for the purpose of step 210 a mechanical perturbation means may comprise an oscillating support on or in which one or more wearable motion sensors can be supported. As another example, for the purpose of step 212 a mechanical perturbation means may comprise a vibrating support or device that can be physically associated with a wearable motion sensor.

FIG. 3 is a flow diagram showing, in schematic form, a method of analysing motion sensing data comprising data from one or more wearable motion sensors and data from a ground reaction force sensing system. At step 310, motion sensing data is provided comprising data from one or more wearable motion sensors worn by a subject and data from a reference motion sensing system comprising a GRF sensing system. At step 312, a first signal is calculated using the vector magnitude of the acceleration recorded by one of the wearable motion sensors. The sensor may be a wearable motion sensor located proximal to the centre of mass of the subject, such as e.g. around the waist. Wearable motion sensors located at other locations on the subject may also be used. In other words, it is advantageous but not necessary for the wearable motion sensor to be located proximal to the centre of mass of the subject. When the system comprises a plurality of wearable motion sensors, the sensor located closest to the subject's centre of mass (e.g. closest to the waist) may be used. The signal is calculated as a function of time, i.e. a vector magnitude is calculated for each of a plurality of time points at which an acceleration measurement recorded by the wearable motion sensor is available. The plurality of time points may be all time points at which the wearable motion sensor recorded acceleration data, or a subset thereof, such as e.g. when the sensor data was down-sampled. The first signal may be the vector magnitude of the acceleration recorded by the sensor, or a quantity that is proportional to this, such as m*∥a∥2, where a is the acceleration of the sensor and m is the mass of the subject or a value derived from the vector magnitude of the acceleration recorded by the sensor, such as e.g. by normalisation. Any normalisation process known in the art may be applied, such as e.g. dividing by the maximum observed value or obtaining a z-score. A z-score may be calculated for an observation x of a plurality of observations as (x−μ)/σ where μ is the average of the plurality of observations and σ is the standard deviation of the plurality of observations. At step 314, a second signal is calculated using the vector magnitude of a force applied to a foot or feet of the subject recorded by the GRF sensing system. For example, the second signal may be calculated by obtaining the sum of the vector magnitude of the forces applied to the feet of the subject recorded by GRF sensing system. The GRF sensing system may therefore comprise a first sensor that measures the force applied to a first foot of the subject and a second sensor that measures the force applied to a second foot of the subject. Alternatively, the GRF sensing system may comprise a single sensor that measures the force applied to a foot of the subject as the subject steps onto the sensor. The first and second signal may be approximately proportional to each other, such as e.g. with a proportionality factor that depends at least in part on the mass of the subject. The proportionality factor does not need to be determined in the present method and may be ignored. In other words, the scale of each of the acceleration vector (from which the first signal is derived) and the force vector(s) (from which the second signal is derived) may be different and may be ignored for the purpose of the present method. Obtaining the second signal may further comprise normalising the vector magnitudes of the force or forces, as explained above. Again this signal is calculated as a function of time, i.e. a sum of vector magnitude of the forces applied to the feet of the subject is calculated at each of a plurality of time points. The plurality of time points may be all time points at which the GRF sensing system recorded data, or a subset thereof, such as e.g. when the GRF sensing system data was down-sampled. At step 316, a time lag is identified that aligns the first signal and the second signal. This may be performed in the illustrated embodiment by determining the cross-correlation of the first and second signals. Thus, the time lag identified may be the time lag corresponding to maximum of the cross-correlation of the first and second signal. At step 318, the identified time lag is applied to the data from the one or more wearable motion sensors or the GRF system to align/synchronise the data from the two systems.

FIG. 4 is a flow diagram showing, in schematic form, a method of analysing motion sensing data comprising data from one or more wearable motion sensors and data from a reference motion sensing system comprising a motion marker tracking system, based on an acceleration signal derived from the two data. At step 410, motion sensing data is provided comprising data from one or more wearable motion sensors worn by a subject and data from a reference motion sensing system comprising a motion marker tracking system. At step 412, a first signal is calculated corresponding to the acceleration of the wearable motion sensor, adjusted to remove the contribution of the acceleration of gravity, in a first reference frame. Step 412 may comprise obtaining a first signal from the wearable motion sensor corresponding to the linear acceleration of the wearable motion sensor (i.e. an acceleration signal that does not comprise a contribution of the acceleration of gravity). Alternatively, step 412 may comprise obtaining a signal from the wearable motion sensor that includes a contribution of the acceleration of gravity, and adjusting this signal to obtain the first signal, as will be explained further below. At step 414, a second signal is calculated corresponding to the acceleration of a marker associated with the wearable motion sensor as measured by the motion marker tracking system, in the first reference frame. The first and second signals are calculated as a function of time. Thus, accelerations are calculated for each signal at each of a plurality of time points for which data from the respective sensors are available (or a subset thereof if the data was down-sampled).

Step 412 can comprise identifying a first rotation matrix (R_{LM}) as a function of time, which aligns the reference frame of the wearable motion sensor (L) with the reference frame of the reference motion system (M), or with any other reference frame that has been selected as the first reference frame. Step 412 can comprise transforming the acceleration of the wearable motion sensor using the rotation matrix. The rotation matrix can be identified using the data from the marker tracking system. The wearable motion sensor may be associated with a plurality of markers detectable by the marker tracking system. The plurality of markers may be positioned so as to not be collinear. In particular, the plurality of markers may be positioned such that a first and second orthogonal vectors are created between the first and second and the second and third markers, respectively. A third orthogonal vector can be obtained from this using a cross-product. The rotation matrix may be a rotation matrix that aligns (i) an orthogonal vector basis associated with the wearable motion sensor in the reference frame of the reference motion sensing system defined using the plurality of markers, with (ii) the reference frame of the reference motion system (M) or any other reference frame for use as a first reference frame that is identifiable by the marker tracking system (e.g. any known orthogonal vector basis). The rotation matrix is also identified as a function of time, so a new orthogonal vector basis may be identified at each time point for which a first and second signals are determined. In general, the choice of any first reference frame in any embodiment describe herein is arbitrary although specific embodiments are described that advantageously simplify the described determinations. Any other reference frame can be used provided that the corresponding rotation matrices can be obtained using the data from the marker tracking system.

Step 414 can comprise determining the position of the marker as a function of time in the first reference frame, and applying one or more noise reduction algorithm to the determined position. For example, the one or more noise reduction algorithm may comprise a low-pass filter (e.g. a Butterworth filter, such as a 6 Hz low-pass 2nd order Butterworth filter) or a smoothing spline, such as a.g. a smoothing spline fitted using generalized cross-validation. Step 414 can comprise determining the second derivative of the (optionally noise-reduced) determined position. The first reference frame can be the reference frame of the marker tracking system. In such embodiments, the position of the marker in the first reference frame may be the position of the marker as recorded by the marker tracking system. If the first reference frame is not the reference frame of the marker tracking system, a rotation matrix may be used to transform the positions measured by the marker tracking system into the first reference frame. The marker used at step 414 may be one of the markers used at step 412 to an orthogonal vector basis associated with the wearable motion sensor in the reference frame of the reference motion sensing system.

At step 416, a time lag is identified that aligns the first signal and the second signal. This may be performed in the illustrated embodiment by determining the cross-correlation of the first and second signals. Thus, the time lag identified may be the time lag corresponding to maximum of the cross-correlation of the first and second signal. Step 412 may comprise determining the magnitude of the linear acceleration of the wearable motion sensor, or the magnitude of the acceleration of the wearable motion sensor adjusted to remove the contribution of gravity. In such embodiments, step 414 may comprise determining the magnitude of the acceleration of the marker as measured by the marker tracking system. Alternatively, step 412 may comprise determining the value of a component of the acceleration of the wearable motion sensor adjusted to remove the contribution of gravity and transformed into the first reference frame. In such embodiments, step 414 may comprise determining the value of the same component of the acceleration of the marker as measured by the marker tracking system in the first reference frame. In other words, the first and second signals aligned at step 416 may be acceleration magnitudes when the acceleration from the wearable motion sensor is a linear acceleration or an acceleration that has been adjusted to remove the contribution of gravity. The first and second signals aligned at step 416 may be acceleration components when the acceleration from the wearable motion sensor is an acceleration that has been adjusted to remove the contribution of gravity and that is expressed in the same reference system as the acceleration of the marker as measured by the marker tracking system. At step 418, the identified time lag is applied to the data from the one or more wearable motion sensors or the marker tracking system to align/synchronise the data from the two systems.

FIG. 5 is a flow diagram showing, in schematic form, a method of analysing motion sensing data comprising data from a wearable motion sensor and data from a marker tracking system (e.g. a motion capture system comprising a marker tracking system), based on a gravity direction derived from the two data. At step 510, motion sensing data is provided comprising data from one or more wearable motion sensors worn by a subject and data from a reference motion sensing system comprising a marker tracking system. At step 512, a first signal is calculated corresponding to the unit gravity direction associated with the wearable motion sensor, in a first reference frame. At step 514, a second signal is calculated corresponding to the unit gravity direction measured by the marker tracking system, in the first reference frame. The first reference frame may be the reference frame of the marker tracking system. The gravity direction in the reference frame of the marker tracking system may be constant and known. It may be a unit vector pointing downwards along the z axis. Thus, when the first reference frame is the reference frame of the marker tracking system, the second signal may be known as a constant default that does not need to be explicitly calculated. The first and second signals are calculated as a function of time. Thus, a unit gravity direction is calculated for each signal at each of a plurality of time points for which data from the respective sensors are available (or a subset thereof if the data was down-sampled).

The first reference frame may be the local reference frame of the wearable motion sensor (L). In such embodiments, determining the second signal can comprise identifying the value of a rotation matrix (R_{LM}) that aligns the reference frame of the reference motion sensing system (M) with the first reference frame (L). The value of the matrix may be determined as a function of time, i.e. at each of the time points where values for the second signal were obtained. The choice of the local reference frame of the wearable motion sensor as a common frame to be used is arbitrary and any other reference frame may be used, such as e,g, in particular the reference frame of the marker tracking system. In such embodiments determining the first signal can comprise identifying the value of a rotation matrix (R_{LM}) that aligns the local reference frame of the wearable motion sensor (L) with the first reference frame (M) as a function of time. Identifying the value of the rotation matrices above may be performed as follows. The wearable motion sensor may be associated with a plurality of markers (e.g. comprising 3 non collinear markers) detectable by the marker tracking system, and the rotation matrix (R_{LM}, R_{ML}) may be a rotation matrix that aligns (i) an orthogonal vector basis associated with the wearable motion sensor in the reference frame of the reference motion sensing system defined using the plurality of markers (e.g. as described above, using a cross product of two orthogonal vectors joining three of the plurality of markers), with (ii) the reference frame of the reference motion sensing system (M), or vice versa (depending on whether R_{LM} or R_{ML} is calculated). The rotation matrix is also identified as a function of time, so a new orthogonal vector basis may be identified at each time point for which a first and second signals are determined.

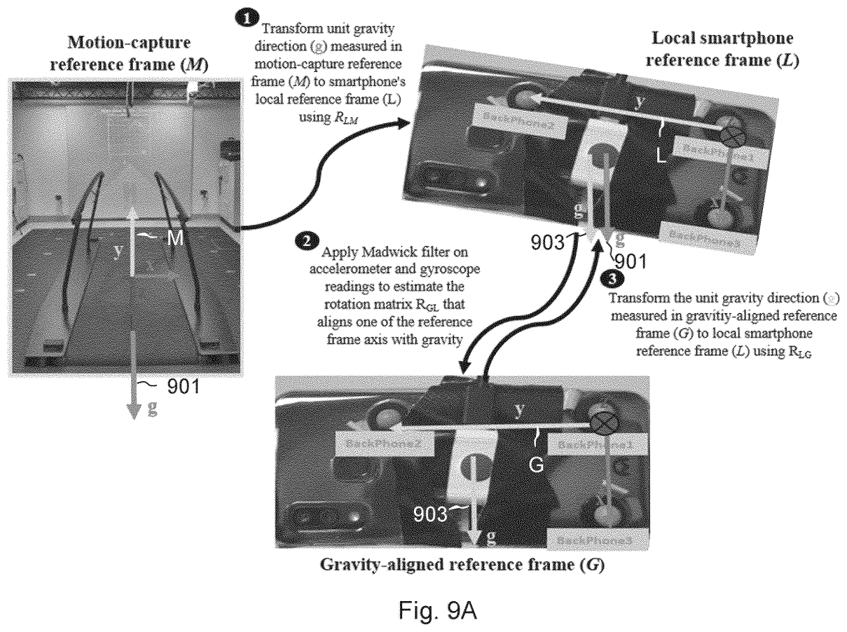

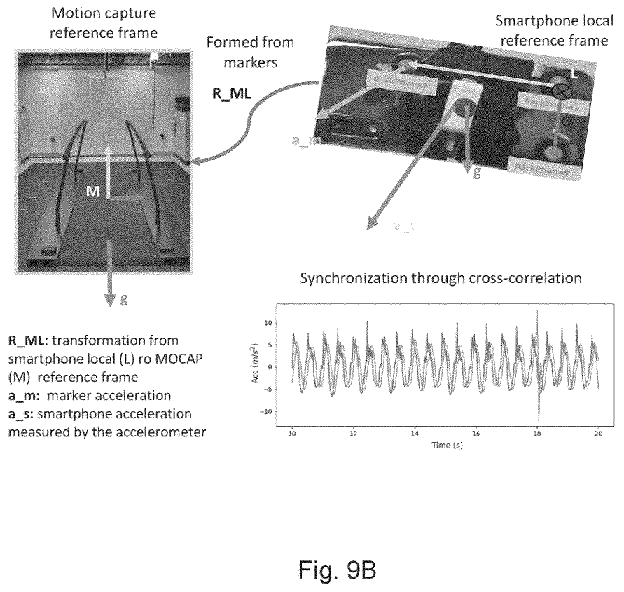

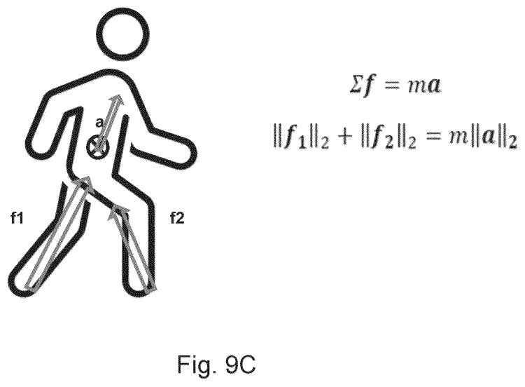

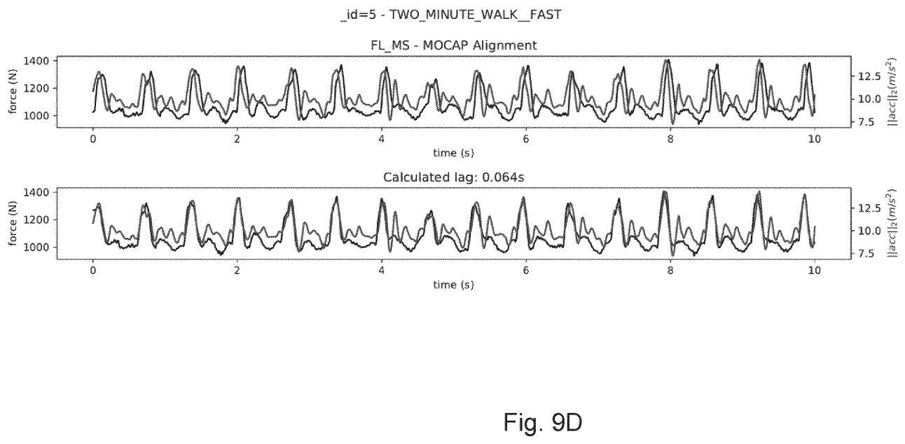

Calculating the first signal can further comprise identifying the value of a second rotation matrix that aligns an axis of the local reference frame of the wearable motion sensor with the gravitational axis (R_{GL}), as a function of time. Any procedure that can identify the gravity direction may be used for this purpose, for example using an orientation filter such as a Madgwick filter, or a Kalman filter. For example, any attitude estimation algorithm available in AHRS: Attitude Heading and Reference Systems may be used (see ahrs.readthedocs.io/en/latest/filters.html), such as a Mahony Orientation Filter, a Madgwick Orientation Filter, an Extended Kalman Filter, the Algebraic Quaterion Algorithm (AQUA), Davenport's q-Method, etc. As the skilled person understands, some of these methods use a different subset of accelerometer, gyroscope and magnetometer data. Thus, depending on whether or not magnetometer data is available, some methods may be preferred over others. For example, some of these methods use accelerometer and gyroscope data, and may optionally make use of magnetometer data (e.g. Madgwick filter, Mahony filter), while other make use of all three types of data (e.g. extended Kalman filter). The first signal can be calculated as

$${R}_{GL}^{T} ( t ) * {[ {v}_{1 G} , {v}_{2 G} , {v}_{3 G} ]}_{G}^{T}$$

where R_{GL}(L) is the value of the rotation matrix that aligns an axis of the local reference frame of the wearable motion sensor with the a gravity aligned reference frame (R_{GL}), as a function of time, and [v_{1G},v_{2G},v_{3G}]_{G} (e.g. [1,0,0]_{G} if gravity is aligned with the red arrow on FIG. 9A by convention) is the unit gravity vector in the local reference frame of the wearable motion sensor rotated to have an axis aligned with the gravitational axis. This reference frame may be defined by an orientation filter or any other procedure that can be used to identify the gravity direction from the sensor data. Its value depends on the axis that is being selected as the axis that is aligned to gravity in the local reference frame of the sensor rotated to have an axis aligned with the gravitational axis. For example, an orientation filter (e.g. a Madgwick filter) may be used that orients signals such that gravity is aligned with the z-axis, in which case the vector [v_{1G},v_{2G},v_{3G}]_{G} may take the value of vector [0,0,1]_{G}. One of [v_{1G},v_{2G},v_{3G}]_{M} will typically have the value of 1 (or −1, depending on the reference frame defined as the local reference frame of the sensor rotated to have an axis aligned with the gravitational axis) and the other two will have the value of 0. Which particular axis is aligned with gravity and has the value of 1 depends on the conventions used when defining the local reference frame of the sensor rotated to have an axis aligned with the gravitational axis. This is a known parameter of the procedure used to define the local reference frame of the sensor rotated to have an axis aligned with the gravitational axis. The second signal can be calculated as

$${R}_{LM} ( t ) * {[ {v}_{1 M} , {v}_{2 M} , {v}_{3 M} ]}_{M}^{T}$$

where R_{LM}(t) is the value of the rotation matrix that aligns the reference frame of the reference motion sensing system with the first reference frame (L in this embodiment), as a function of time, and [v_{1M},v_{2M},v_{3M}]_{M} is the unit gravity vector in the reference frame of the reference motion sensing system. The value of the vector [v_{1M},v_{2M},v_{3M}]_{M} depends on the conventions used by the reference motion system. One of [v_{1M},v_{2M},v_{3M}]_{M} may have the value of −1 (or +1, although −1 is more likely as gravity is typically represented as pointing downwards in the reference frame of a reference motion system) and the others may have the value of 0. Which particular axis is aligned with gravity and has the value of −1 depends on the conventions used by the reference motion system to record data. This is a known configuration parameter of the reference motion system. In embodiments, the vector [v_{1M},v_{2M},v_{3M}]_{M} may have the value vector [0,−1,0]_{M}. This was the value corresponding to the configuration of the reference motion system used in the examples of the present disclosure.

A gravity-aligned reference frame (G) is an orthogonal reference frame that has an axis (e.g. z-axis) aligned with the direction of gravity.

At step 516, a time lag is identified that aligns the first signal and the second signal. This may be performed in the illustrated embodiment by determining the cross-correlation of the first and second signals. Thus, the time lag identified may be the time lag corresponding to maximum of the cross-correlation of the first and second signal. At step 518, the identified time lag is applied to the data from the one or more wearable motion sensors or the reference motion sensing system to align/synchronise the data from the two systems.

### Applications

The methods described herein find use in the context of analysing motion sensing data in a clinical context, such as for example to assess, monitor, diagnose, provide a prognosis or treatment recommendation for a subject with a neurologic disorder. In particular, the methods described herein may be used to analyse motion sensing data to quantify one or more gait features. As used herein, the term “gait feature” refers to a parameter that characterises a subject's gait.

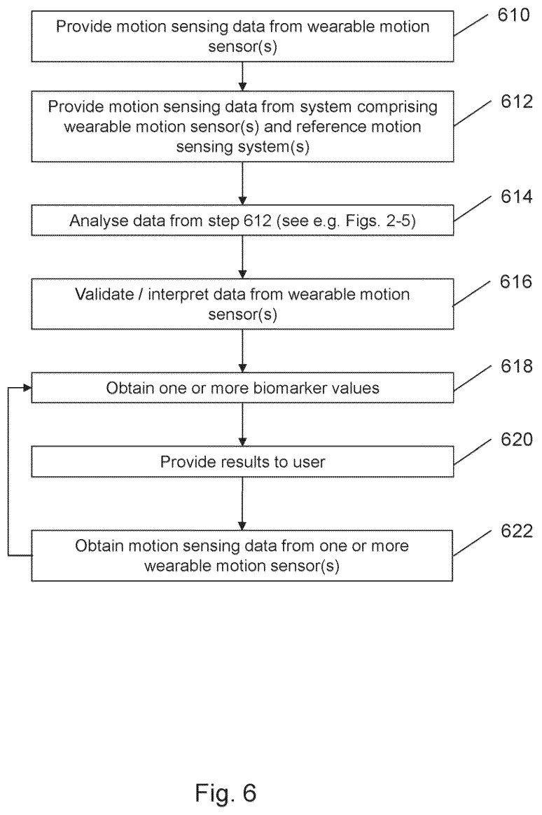

FIG. 6 illustrates an example of a method of analysing motion sensing data from a subject, in a clinical context. At step 610, motion sensing data previously obtained from one or more wearable motion sensors associated with a subject is provided. The data may have been obtained while the subject was performing one or more motion-based tests (e.g. one or more gait and/or balance tests) in a non-clinical environment (also referred to as “remote” or “unsupervised” environment. At step 612, motion sensing data is obtained from one or more wearable motion sensors and one or more reference motion sensing systems. The data is associated with the subject, and is advantageously obtained while the subject is performing one or more motion-based tests (e.g. one or more gait and/or balance tests) in a clinical/supervised environment. The motion sensing data obtained at step 612 is obtained from both the wearable motion sensors and the reference motion sensing systems for the same movement occurrence (i.e. the data comprises matched data form wearable motion sensor(s) and reference motion sensing system(s)). In other words, the motion sensing data obtained at step 612 is obtained from a system comprising both one or more wearable motions sensors and one or more reference motion sensing systems. The one or more wearable motion sensors may be or may comprise the same sensors or types of sensors that were used to obtain the data at step 610. By contrast, the data obtained at step 612 does not comprise data from a reference motion sensing system (i.e. no matched data). The methods described herein can be used to validate or interpret the data from step 610 by joint analysis of the data from step 612.

At step 614, the data obtained at step 612 is analysed using any method described herein. At step 616, the results of step 614 are used to validate and/or interpret the motion-sensor data obtained from the wearable motion sensors obtained at steps 610 or 612. This may comprise identifying one or more motion-based tests and/or motion features derived therefrom (e.g. gait features) that can be reliably quantified using the wearable motion sensors by comparison with corresponding features derived from synchronised data from one or more reference motion sensing systems. This may comprise identifying one or more motion-based tests and/or motion features derived motion sensing data (e.g. gait features) that can be reliably quantified using the wearable motion sensors by comparison with corresponding features derived from synchronised data from one or more reference motion sensing systems. Thus, step 616 may comprise quantifying one or more motion features in one or more tests and identifying motion features that can be reliably quantified (whether across all tests or in specific tests). Instead or in addition to this, step 616 may comprise interpreting the data from the wearable motion sensors obtained at steps 610 or 612 based on the results of step 614, such as e.g. by identifying and correcting for systematic biases that may be present in the wearable motion sensor data.

At step 618, the results from step 616 are used to obtain one or more biomarker values derived from the data from a wearable motion sensor, such as that obtained at step 610. For example, the biomarker values may be the values of one or more motion features derived from the wearable motion sensor data that were identified at step 616 to be reliably quantified, and that are also known to correlate with a clinically relevant parameter. As another example, the one or more biomarker values may comprise a clinically relevant parameter obtained by mathematical analysis of the data from a wearable motion sensor (e.g. by machine learning, see e.g. Creagh et al. 2022 which described the use of machine learning analysis of smartphone inertial sensor data to classify patients between categories comprising two MS disease severity categories and a healthy category). A clinically relevant parameter may be a prognosis, a diagnosis (e.g. presence/absence of a particular disease or disorder), a disease progression metric, a treatment response prediction, a symptom/disease severity metric, etc. Thus, also described herein are methods of providing a prognosis, diagnosis, disease progression metric, or treatment recommendation for a subject, and methods of treating and/or monitoring a subject, the methods comprising providing wearable motion sensor data obtained from the subject and analysing this data using any embodiment of any method described herein.

At optional step 620, one or more results of steps 614, 616 or 620, or information derived therefrom, may be provided to a user, for example through a user interface. The results may comprise one or more biomarker values, a clinically relevant parameter, information identifying a particular test, sensor or motion feature as being reliably or not reliably quantified using a wearable motion sensor, etc. Optionally, the method may further comprise obtaining 622 further motion sensing data from one or more wearable motion sensors associated with a subject, where the data may be obtained while the subject is performing one or more motion-based tests in a non-clinical environment, and repeating step 618 (and optionally 620).

The methods of the present disclosure find particular use in the context of gait analysis. Gait analysis is the study of a subject's motion, and in particular their pattern of walking. The subject is typically a human subject, although motion analysis can also be performed for animals. Gait analysis can comprise quantifying a plurality of parameters (also referred to as “gait features”), which can be selected from: step length, stride length, cadence, speed, dynamic based, progression line, foot angle, hip angle, and squat performance. Abnormal or pathological gait can be identifying by changes in one or more of the above gait features compared to a normal (healthy) control reference value. Thus, the one or more biomarker values obtained at step 618 of the method of FIG. 6 may be the values of one or more gait features or values derived therefrom.

The methods of the present disclosure find particular use in the context of analysis of motion sensing data from a subject who has been diagnosed as having or being likely to have a neurological disorder. A neurological disorder is typically a disorder that affects gait and/or balance. A neurological disorder can be selected from: neuromuscular disorders (such as e.g. multiple sclerosis, MS, muscular dystrophy, myopathy, peripheral neuropathy, etc.), a neurodegenerative disorder (such as e.g. amyotrophic lateral sclerosis, MS, Parkinson's disease, Alzheimer's disease, Huntington's disease, etc.), a brain or other central nervous system cancer, etc. In embodiments, the neurologic disorder is MS.

The following is presented by way of example and is not to be construed as a limitation to the scope of the claims.

### EXAMPLES

In the study ‘Validation of smartphone-based assessments in multiple sclerosis’ (ISRCTN15993728), the inventors aim to establish the analytical and clinical validity of the smartphone-based gait and balance tests included in the Floodlight technology. In particular,, establishing analytical validity is a critical step in ensuring that the measures derived from these tests are accurate and precise. This includes comparing the sensor data collected with the Floodlight technology against data collected simultaneously with reference measurement systems (e.g., motion-capture systems or ground reaction forces [GRF] plates). These comparisons pose several technical challenges. One is to guarantee that the information on test type (i.e., test identifier and test condition) has been correctly entered on the different measurement systems. This ensures that subsequent analyses only compare data collected during the same test execution. A second challenge is to accurately synchronize sensor data collected with the different systems.

In these examples, the inventors describe new solutions for both validating metadata such as test type and for synchronizing sensor data. Their effectiveness is illustrated using data obtained with the Floodlight technology and a motion-capture system. In particular, Example 1 describes the data and system used, and demonstrates the use of a new method for validating metadata and motion-capture onset detection. Example 2 describes and demonstrates the use of new methods for synchronising wearable motion sensor data (any IMU sensor, in the present case smartphones) with a motion-capture system comprising cameras. Example 3 describes and demonstrates the use of new methods for synchronising wearable motion sensor data (any IMU sensor, in the present case smartphones) with a motion sensing system comprising a ground reactive force sensor (multiaxial force plates embedded in a treadmill).

### Example 1—Annotation and Onset Detection of Smartphone Inertial Measurement Unit and Motion-Capture Data Collected in a Laboratory Setting to Study Gait and Balance

The present and following examples describe the development and use of new methods for annotation and synchronization of smartphone inertial measurement unit and motion-capture data collected in a laboratory setting to study gait and balance. However, the methods are equally applicable to any environment in which data from such sensors is to be jointly analysed.

### Study Design and Participants

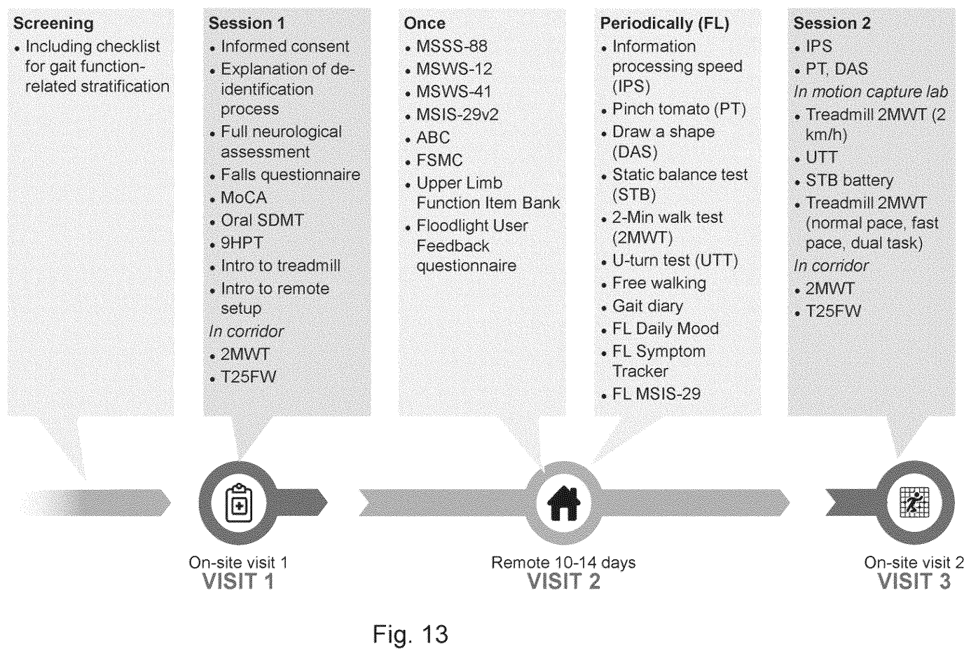

The protocol for the study ‘Validation of smartphone-based assessments in multiple sclerosis’ (ISRCTN15993728) is illustrated on FIG. 13. This includes a recruitment and screening period (grey) and an observation period (blue), which includes two on-site visits in a gait laboratory (indicated as “Session 1” and “Session 2”) and an unsupervised, remote testing period for 10-14 days in between. A motion-capture system is used during the second on-site visit, and data from this visit are presented in these examples using an interim data cut. Both people with MS (PwMS) and healthy controls (HC) are recruited (age and sex-matched). All participants provided signed informed consent.

During the second on-site visit, all participants perform several gait and balance tests. These include the Two-Minute Walk Test (2MWT) [Montalban et all 2022, Bourke et al. 2020], U-Turn Test (UTT) [Cheng et al. 2021], and Static Balance Test (SBT) battery. There are four separate 2MWT conditions, or walking tasks, each lasting two min: 1) fixed speed (at 2 km/h), 2) self-paced (at normal pace for the participant), 3) fast-paced (as fast as the participant can walk safely), and 4) dual-task (self-paced condition with a simultaneous cognitive task consisting of serial subtractions of 7 starting from 200). The UTT instructs the participant to walk back and forth for 60 s while performing U-turns roughly 3 or 4 m apart. The SBT battery consists of five test conditions, each performed twice and each lasting 30 s: 1) natural stance with feet apart and eyes open (“eyes open”); 2) natural stance with feet apart and eyes closed (“eyes closed”); 3) parallel stance with feet together and eyes open; 4) full tandem stance with eyes open; and 5) single foot stance with eyes open.

A total of 32 PwMS and 9 HC were included in the interim data cut used for this analysis. Their baseline demographics and disease characteristics are summarized in Table 1. Together, they contributed towards 590 sensor-based tests (2MWT: 159 test executions, UTT: 41 test executions, SBT: 390 test executions).

TABLE 1
Baseline demographics and disease characteristics

| | PwMS (n = 32) | | HC (n = 9) | |
|---|---|---|---|---|
| Variable | | | | |
| Age, years, mean (SD) | 59 | (8) | 54 | (6) |
| Female, n (%) | 25 | (78) | 5 | (56) |
| T25FW, s, mean (SD) | 6.30 | (2.83) | 3.74 | (0.26) |
| BBS, mean (SD) | 11.58 | (2.35) | 9.47 | (1.64) |
| Diagnosis | | | | |

| | | | |
|---|---|---|---|
| RRMS, n (%) | 23 | (72) | — |
| SPMS, n (%) | 3 | (9) | — |
| PPMS, n (%) | 6 | (19) | — |
| EDSS, mean (SD) | 4.75 | (1.43) | — |

### Floodlight Technology and Reference Measurement System

The Floodlight technology consists of smartphone sensor-based tests of gait and balance as well as other functional domains affected by MS [Montalban et al. 2022]. In this study, it records continuous accelerometer, gyroscope, and magnetometer data to compute a wide range of gait and balance characteristics. In the present examples, the magnetometer data was not used. However, magnetometer data may be used in embodiments of the disclosure to determine the orientation of a wearable motion sensor. The sampling frequency is 50 Hz (accelerometer, gyroscope, and magnetometer data are simultaneously and continuously recorded from all six smartphones at a sampling frequency of 50 Hz). The study participants perform the gait and balance tests while carrying six Samsung Galaxy A40 smartphones in six different wear locations (the right and left front pockets; the central front at the waist; the left and right back pockets; and the back waist) attached to customized shorts or carried in an adjustable belt. These smartphones came with the Floodlight technology preinstalled (Floodlight GaitLab v1.0.6 or newer). Two additional smartphones are available to the experimenters: the “timer” and “master” smartphones. The timer smartphone is used by the experimenters to record the metadata associated with a test (test type and the timestamps marking the start and end of each test execution). This information is used to segment the continuous smartphone sensor data (timestamps) and annotate these segments (test type). The master smartphone is used to synchronize the smartphones with the motion-capture system. The master smartphone is not handled by the operator and remains static during data acquisition, as this improves the detection of the synchronization signal.

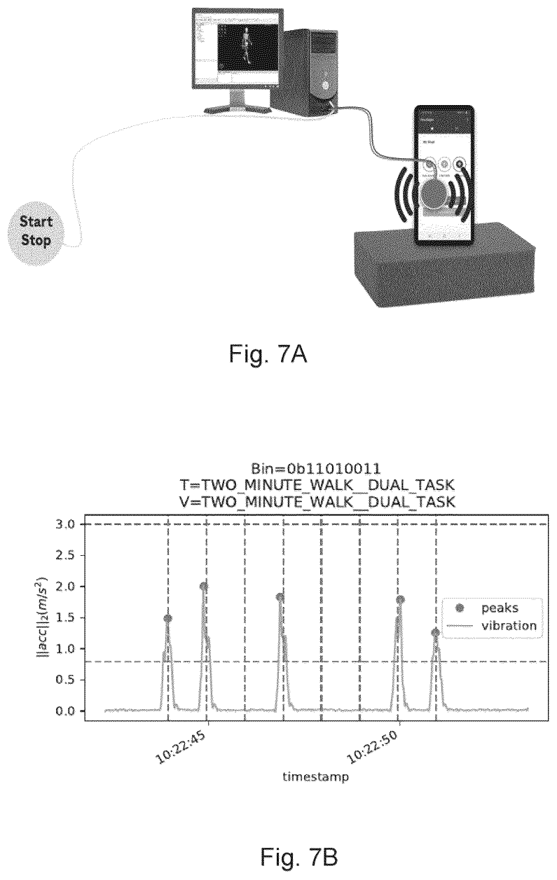

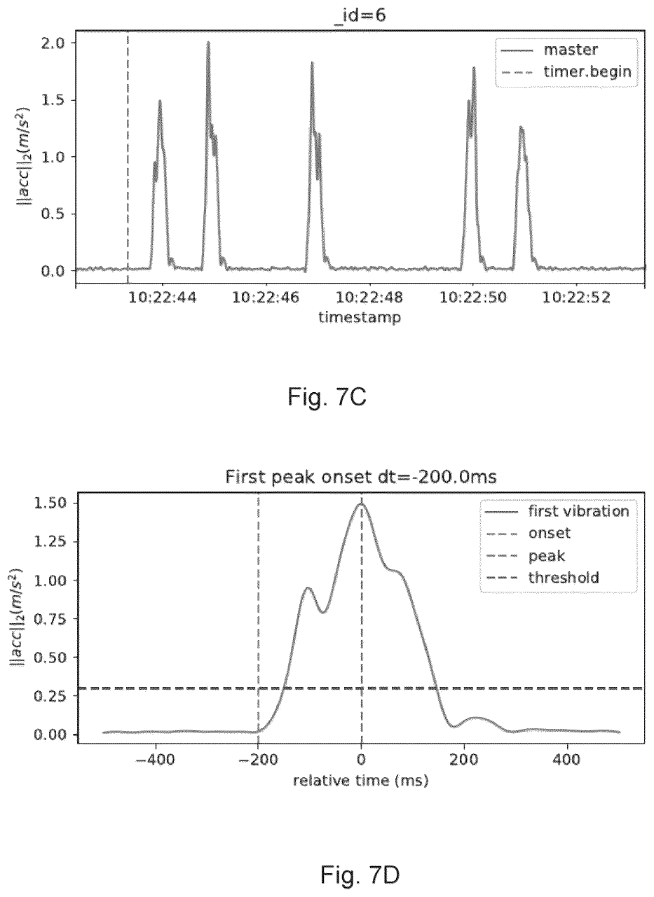

The reference measurement system used in these analyses is a Vicon™ motion-capture system consisting of 12 infrared cameras (Vicon Vero cameras), which capture the signals from 26 reflective markers attached to the participant's clothing and 3 reflective markers attached to each of the six smartphones worn by the participants, and a split-belt treadmill (Motek, Netherlands) that is leveled with the ground. Multiaxial force plates (ForceLink R-Mill force plates, Motek, Netherlands) are embedded in the treadmill and record GRF. The infrared camera and treadmill data are internally synchronized with each other. The sampling frequency is 100 Hz for the infrared cameras and 1000 Hz for the treadmill. Each motion-capture recording is initiated and stopped by the experimenter pressing a start/stop button (green button FIG. 7A) of the reference measurement system.

Additional sensing systems used in the study are GaitUp IMU sensors and pseudonymized video recordings (data not shown). The GaitUp IMU sensors may be treated in the same way as the smartphones and may be synchronized in the same manner.

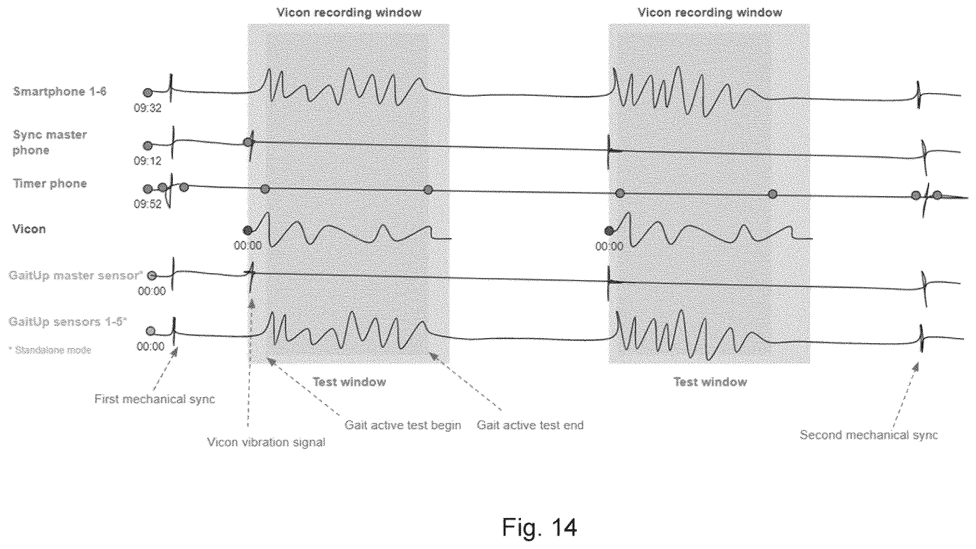

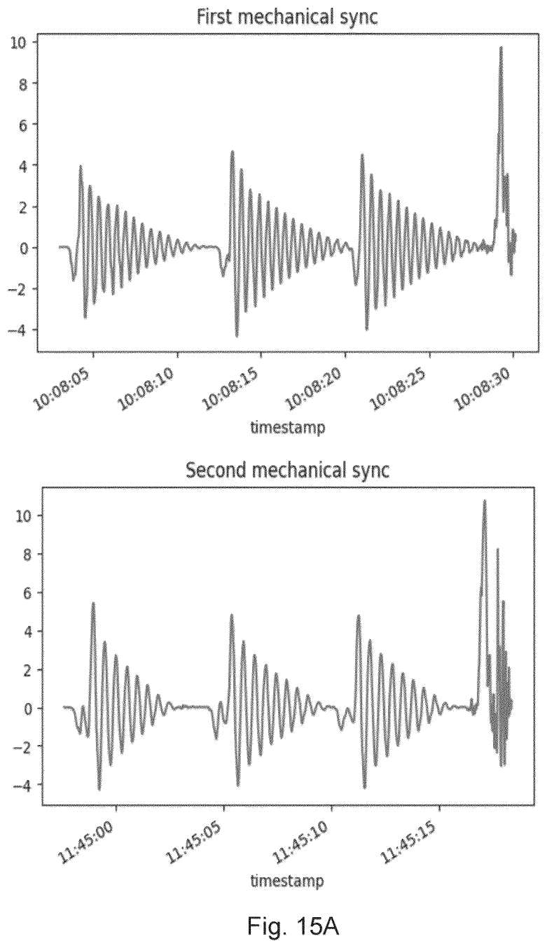

An example of the signals measured from all of these sensors is shown on FIG. 14, which also illustrates the signals used for synchronization as described in the present example.

### Synchronization of Inertial Measurement Units

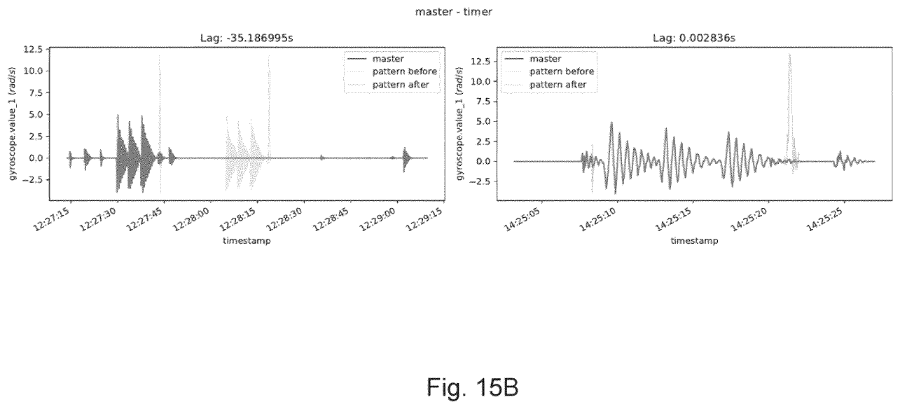

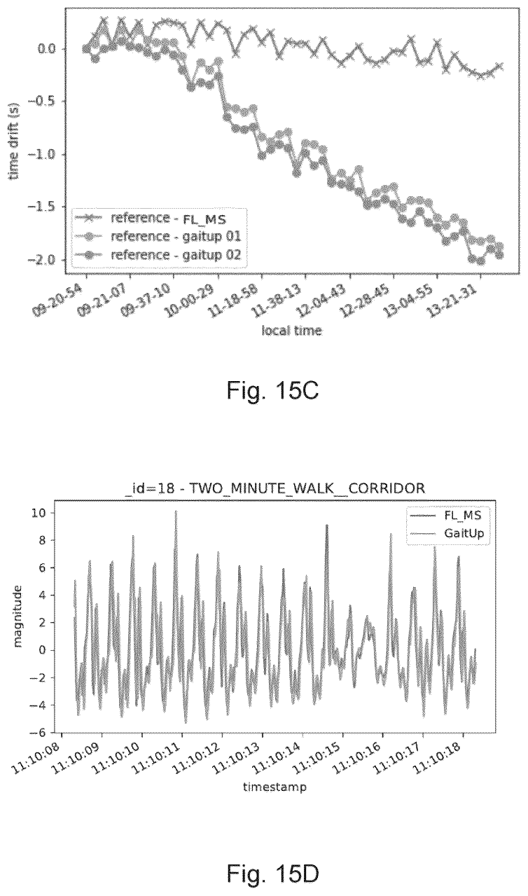

The smartphones are synchronized with each other by two mechanical perturbations at the beginning and end of the second on-site visit, respectively. They are placed in a half-tube that, while rocking, produces a distinct pattern easily identified in the gyroscope signal (see FIGS. 15A, 15B). The first mechanical perturbation is used to align the timestamps of all smartphones by selecting one of the devices as a reference (e.g., the master smartphone) through cross-correlation. The second is used to correct for possible clock drift. In particular, this synchronization process operates as follows. In a first step, the 8 smartphones and the Gaitup sensors are placed on a half cylinder which is then tilted to create a movement that is easily identified by the gyroscopes (FIGS. 15A, 15B). The first and last mechanical perturbations are automatically detected in the gyroscope data. This is used to calculate a lag and drift between the master smartphone (“vibration phone”) and the timer smartphone, by aligning the gyroscope data from these devices using cross-correlation. The lag and drift information is further used to adjust timer phone's active_test table timestamps into the master's time frame. The gyroscope data is also used to calculate a lag and drift between the master smartphone and the other smartphone devices (also by cross correlation), and to adjust the signal from these phones.

As all eight smartphones have been synchronized with each other, the following analysis only uses the smartphone worn at the waist level near the participant's center of mass (CoM). Any annotation and/or synchronization adjustment identified using the data from this smartphone can be used to annotate and/or synchronize the other smartphones.

The GaitUp devices are synchronized internally. However, this process sometimes fails so all GaitUp devices are also subjected to the two mechanical perturbations described. The data from the GaitUp master device is synchronized with that of the smartphones by cross correlation of the gyroscope signals from the master smartphone and the master GaitUp. FIG. 15C shows that the GaitUp sensors drift similarly relative to each other but not necessarily the same way as the smartphone sensors. In this experiment, the timestamps of two GaitUp sensors and a smartphone were compared with respect to a reference pulse generator that has a very accurate clock. The results showed that the smartphone does not drift much (FIG. 15C, top data series), whereas the two GaitUp devices drift (approximately linearly) with respect to the reference (FIG. 15C, two downward sloping data series).

All offset and drift corrections performed assumed a linear drift (i.e. drift was corrected by linear scaling of the time signal). This is because the drift was observed to be linear. However, the same methods can be applied to correct a nonlinear drift using a non-linear function.

The alignment of test data was then manually inspected. FIG. 15D shows that at an active test level the smartphone and GaitUp time series are synchronized confirming the synchronization process done previously before segmenting and exporting the active tests. FIG. 15D therefore confirms that both alignment and drift correction worked as expected,

### Automatic Validation of Metadata and Motion-Capture Onset Detection

The wide range of different gait and balance tests and measurement systems included in this study increases the risk of false annotation of sensor data. Such errors could lead to inadvertently comparing sensor data recorded during different test executions with each other. Hence, the inventors implemented a process to validate that the test type has been correctly entered on the different measurement systems. For this, they developed a fully automatized, mechanical solution, which requires only simple signal processing procedures and a low-cost vibrating motor. Each time a motion-capture recording is initiated, a short binary sequence of pulses consisting of ‘0s’ and ‘1s’, which are spaced by 1 s, is sent to a programmable vibrating motor (Seeed Studio 316040001 Mini Vibration Motor, Seeed Studio, China) mechanically attached to the master smartphone (FIG. 7A). The vibrating motor starts to vibrate for 0.3 s followed by 0.7 s rest each time it receives a ‘1’. This vibration train is subsequently detected with the master smartphone's accelerometer. To mark the start and end of each vibration train, each vibration pulse sequence starts and ends with ‘11’, respectively. The test type is encoded by the middle four bits of the sequence, thus allowing us to encode 16 different test types (e.g., 0000, 0001, 0010, . . . , 1111). For example, the vibration pulse sequence ‘11010111’ shown in FIG. 7B encodes the test type assigned to ‘0101’ (i.e., ‘UTT’).

Decoding this information involves several steps (FIG. 7B). First, the vibration signal is automatically segmented by localizing the vibration train in the master smartphone's accelerometer data (i.e., when the accelerometer reading is above a preset threshold [lower dashed line in FIG. 7B] but also below a second threshold [upper dashed line in FIG. 7B]). The first and second thresholds are identified experimentally. As illustrated on FIG. 7C, the vibration train is expected to be localized in the vicinity of the timer start timestamp. Thus, a window of time around the timer start stamp may be searched. An appropriate window of time to search may be defined depending on the circumstances such as e.g. the expected amount of time between a timer start timestamp and movement data associated with movement of a subject (such as e.g. a subject performing an active test). For example, a window of 60 seconds after the start time may be searched. In the present examples, because the vibration was associated with the master sensor which is not moved during data acquisition, no other signal satisfied the criteria above and thus it was not necessary to define a specific window of time to search. In embodiments an entire movement data set may be searched rather than a selected window of such a dataset. In embodiments, one or more segments that comprise a pattern of signals that may correspond to the vibration train may be identified (such as e.g. a plurality of peaks separated by multiples of a time period t). When a plurality of segments are identified, a single segment may be selected as the first of the plurality of segments, or a segment that comprises a pattern of signals that most closely match an expected signal corresponding to the vibration train. Next, the peaks (marked by blue circles in FIG. 7B) within this segmented signal are automatically detected. Peaks are detected as local maxima that satisfy a plurality of constraints comprising a minimum height constraint and a time between peaks constraint (minimum distance between consecutive peaks). For example, all local maxima may be identified, then local maxima that do not satisfy a minimum heigh constraint may be removed, then the smallest peaks may be iteratively removed until all remaining peaks satisfy a minimum distance between peaks constraint. These peaks correspond to the ‘1s’ in the binary vibration pulse sequence. As we know a priori at which exact time points a ‘1’ or ‘0’ occur (green dashed lines in FIG. 7B spaced by 1 s), the information on test type can be decoded by determining at which of these time points a peak in the accelerometer magnitude signal occurred. The information on test type is then validated by comparing this automatically decoded information with the manually entered information with the timer smartphone.

Additionally, as illustrated on FIG. 7D, detection of the first peak's onset in the vibration train is also used to mark on the master smartphone's data time series the onset of the motion-capture recording, thereby synchronizing the two measurement systems with each other (vibration-based method). This is also used to synchronize the smartphones and the GaitUp sensors. As shown on FIG. 15D, this was able to both align time series and correct for drift.

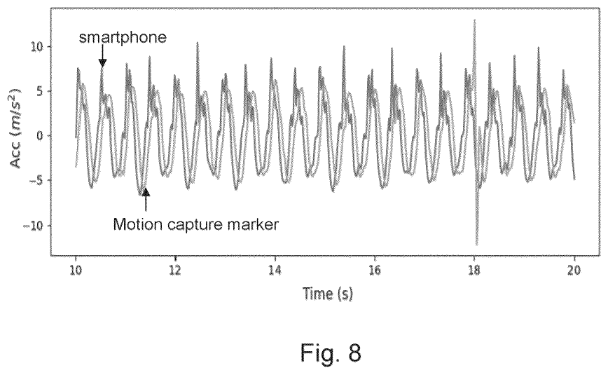

The vibration train initiated by the motion-capture system was used to indicate on the master smartphone the start of each motion-capture recording, which in turn was used for an initial synchronization between the smartphones worn by the study participants and the motion-capture system (vibration-based method). However, even after this initial synchronization, a small lag can be observed between the accelerometer readings of the two measurement systems that is independent of test type (FIG. 8). Hence, additional, post-synchronization correction methods were developed to optimally synchronize the two measurement systems (described in Examples 2 and 3 below). Note that this remaining small lag may not be present in all systems and it may therefore in some cases be sufficient to perform an initial synchronization as described in the present example.

### Example 2—Synchronization of Smartphone Inertial Measurement Unit and Motion-Capture Data (Cameras)

In this example, the inventors used the approach described in Example 1 to analyse motion sensing data. They identified that inertia of the vibrating motor or delays introduced by the system used for creating the vibration pulse sequence may potentially lead to small, non-controlled lags between the smartphone and motion-capture sensor data. If present, such lags could be estimated through cross-correlation of corresponding signals measured with both systems and hence corrected. In particular, the inventors noted that when comparing gait events such as heel strike events detected with the two measurement systems, there was a systematic bias in the time points at which these events were detected by the two measurement systems. The motion-capture system detected heel strike events on average (SD) 47.4 (35.6) ms earlier than the smartphones. The inventors explored methods to address this, described in the present and following examples. All methods are applied to each test execution separately. Given the short duration of each test execution (2 min or less), any potential clock drift between the measurement systems was considered negligible. In both Examples 2 and 3, the methods described were applied after initial synchronization with the vibration-based method. This had the advantage of additionally including the data annotation feature. However, the methods described in Examples 2 and 3 could also be used without such an initial synchronization.

### Acceleration-Based Method

The acceleration-based method involves cross-correlating the acceleration of the smartphone with the acceleration of one of the Vicon reflective markers attached to the smartphone. As the smartphone's acceleration also measures the acceleration due to gravity, additional transformations are used to make the two measurements comparable. The three makers attached to the smartphone form a three-dimensional orthogonal vector basis that is used to transform the smartphone's acceleration from its local reference frame to the motion-capture reference frame. After removing the contribution of gravity, the two accelerations can be compared (e.g., y-axis) as they are both expressed in the same reference frame. The marker's acceleration is calculated using finite differences after filtering the marker position by a zero-lag 6 Hz low-pass 2nd order Butterworth filter. In other words, the marker position signal was filtered to remove high frequency noise and then finite differences was applied to this) dy/dt=(y(t+dt)−y(t))/dt) to get the derivative. Alternatively, the marker's acceleration may be calculated after filtering the marker position using GCV splines, or by using GCV splines after differentiation of the marker position.

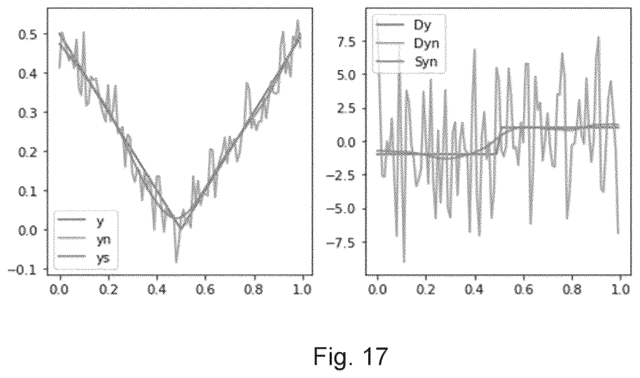

A Butterworth filter is a signal processing filter that is designed to be maximally flat in the passband and roll off to zero in the stopband. A second order filter is one that decreases at −12 dB per octave. A first order filter rolls of at −6 dB per octave and a third order filter rolls off at −18 dB per octave. A lowpass Butterworth filter removes the high frequency content of the signal. This was used here because the meaningful signal associated with gait is expected to be within a frequency band below 6 Hz, so frequency content above that could be excluded. Depending on the specific type of motion data to be analysed, passbands extending to e.g. 10 Hz may be used. A filter to remove high frequency signals (e.g. above 6, 8 or 10 Hz) is advantageous in this method because the method involves two differentiations of the signal, which would amplify noise that might be present. The present inventors found a simple noise filter removing high frequencies to be sufficient for the differentiated signal to have enough signal to noise ratio for the purpose of the present methods. However, other methods for addressing the problem of noise can be used and have been investigated such as Kalman smoothing filter, total variation regularization derivatives, and generalized cross-validated splines. For example, the inventors tested a generalized cross-validated spline method, in which a smooth spline is fitted using a penalty term (estimated internally) to prevent overfitting. This is illustrated on FIG. 17, which shows the shape of a raw noisy signal (left) or its derivative (right) (orange oscillating curve), the theoretical shape of the derivative and second derivative (blue curve forming a V or step signal, i.e. noise-free signal that generated the noisy, oscillating raw signal) and the spline function calculated using a cross-validated spline method (smooth green curve following the theoretical curve) fitted to the raw noisy signal (left) and its derivative (right). This shows that such a method is able to correctly approximate the true signal (blue) based on a noisy version of the signal (orange curve). A Kalman filter fits a dynamic model to the observations (data) while learning the noise in this data to estimate an unobserved state. In this case, a very simple dynamic system (a point that accelerates) may be used, and the Kalman filter may be used to estimate the first and second derivatives as states from the filter. Total variation regularization derivatives is a method for differentiating a function specified by noisy data, described in Chartrand R. (ISRN Applied Mathematics, Vol. 2011, Article ID 164564, 2011)

The acceleration-based method is illustrated in more detail on FIG. 9B. As illustrated, the method involves the following steps:
1. The accelerometer data from the smartphone and the position of the three markers on the phone are filtered (2nd Order Butterworth zero-lag 6 Hz) and resampled to a common sampling frequency. Note that any of the acceleration directions can be used. The acceleration data is filtered to remove high frequency noise and smooth the signal to improve the comparison at step 5 below. The marker position data is filtered to remove high frequency noise which can have a very strong impact on the signal once differentiated.
2. The position of one of the 3 markers (measured by the reference motion sensing system, any of the 3 markers can be used, or indeed any marker attached to the smartphone) in the reference system reference frame M is differentiated using finite differences, to obtain the marker's acceleration in the reference system M.
3. The position of the three markers is used to obtain a reference frame L that is used to transform the smartphone acceleration into the motion capture reference frame M. In particular, a rotation matrix that aligns local smartphone reference frame L with the reference frame M of the reference system (R_{LM}) is calculated. This is calculated by computing an orthogonal vector basis using the three markers attached on the smartphone whose position is measured in the reference frame M by the motion capture system.
4. The contribution of gravity to the acceleration signal is removed from the smartphone measurements once transformed into reference frame M where gravity direction is known to obtain the linear acceleration.
5. The acceleration obtained at step 4 (smartphone acceleration signal transformed in reference frame M and with gravity removed) is compared with the acceleration of the marker (measured in reference frame M, obtained at step 2) through cross-correlation to determine the lag.

Note that if the smartphone acceleration signal is the linear acceleration (i.e. if the smartphone measures linear acceleration), then the magnitude of this can be directly compared with the magnitude of the acceleration of the marker at step 5 (without needing steps 3 and 4 above) since the acceleration magnitude is invariant to the coordinate system.

In other words, if the IMU measures linear acceleration without the contribution of gravity, then: (i) the marker position can be filtered (optionally resampled) and differentiated to obtain a marker acceleration, from which an acceleration magnitude is calculated, and (ii) the IMU linear acceleration (optionally filtered and/or resampled) can be directly used to calculate an acceleration magnitude. The results of (i) and (ii) can the be cross-correlated.

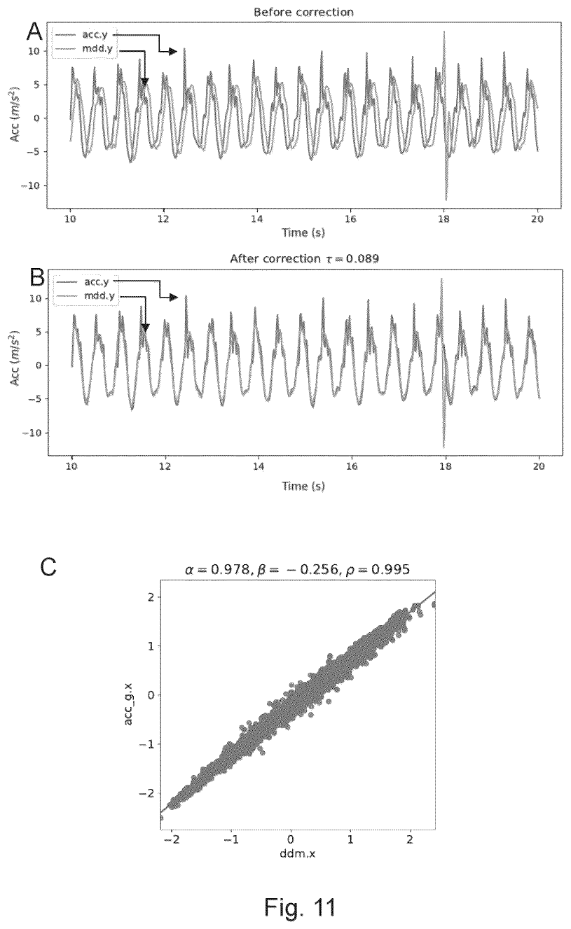

The cross-correlation between the acceleration of the smartphone (blue curve in FIGS. 11A and 11B) and that of the Vicon markers (orange curve in FIGS. 11A and 11B) demonstrates an improved temporal alignment between the two signals after applying this post-synchronization correction. The scatterplots in FIG. 11C indicate a high degree of agreement between the two acceleration signals during walking tasks.

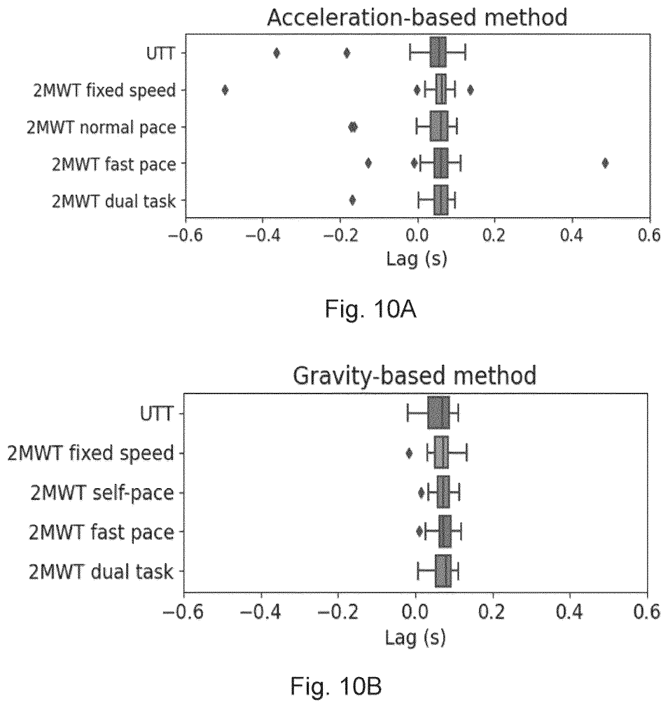

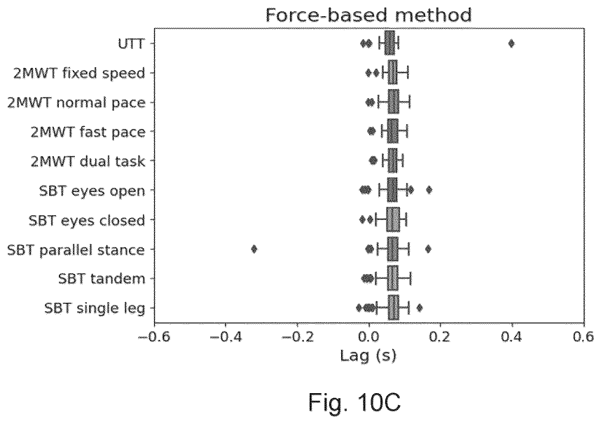

With the acceleration-based method, the lag between the acceleration of the smartphone and the acceleration calculated for the Vicon markers attached to the smartphone across all study participants in terms of percentiles (25%, 50%, 75%) is [0.042, 0.061, 0.077] s for the Floodlight gait tests (2MWT and UTT) and [-0.136, 0.023, 0.089] s for the SBT battery (FIG. 10A). Moreover, the distribution of the detected lag indicates that this method was able to find a consistent lag across all 2MWT/UTT conditions and study participants.

### Gravity-Based Method

The gravity-based method is illustrated on FIG. 9A. The method involves cross-correlating the unit gravity direction in the motion-capture reference frame (M, which is always constant and known) with that estimated by the smartphones, worn by the study participants, in their respective local reference frames (L). One of the challenges of this approach is that the comparison must be done in the same reference frame. By convention, the inventors select the smartphone's local reference frame (L) as a basis for this comparison (although the motion capture reference frame or any other known reference frame could be used instead). Hence, the unit gravity direction measured in the motion-capture reference frame (M) must be transformed to the smartphone's local reference frame (L) (step 1 in FIG. 9A). This transformation is computed by calculating a rotation matrix (R_{LM}), which transforms a vector expressed in reference frame M into the vector in reference frame L using the three Vicon markers attached to each smartphone worn by the study participants. These markers are attached such that they form an orthogonal vector basis (their positions are measured in M). As gravity points in the negative y-axis direction in motion-capture reference frame (M), one can transform the unit gravity direction in the motion-capture reference frame into a gravity direction in the reference frame L (g_{mocapL}) by computing:

$${g}_{mocapL} ( t ) = {R}_{LM} ( t ) * {[ 0 , - 1 , 0 ]}_{M}^{T}$$

A second challenge is to estimate the smartphone's unit gravity direction. The inventors first applied a Madgwick filter (Madgwick, 2010) (or any other known orientation filter—where an orientation filter is an algorithm that determines an estimate of orientation of a IMU using gyroscope and accelerometer data, and sometimes also magnetometer data) to the smartphone's accelerometer and gyroscope readings to estimate the rotation matrix R_{GL} that is needed to align one axis of the smartphone's local reference frame L with a gravity aligned reference frame G (step 2 in FIG. 9A). The smartphone's gravity in L (gsmartphoneL) was thus estimated by computing (step 3 in FIG. 9A):

$${g}_{smartphoneL} ( t ) = {G}_{LG} ( t ) * {[ 1 , 0 , 0 ]}_{G}^{T} = {R}_{GL}^{T} ( t ) * {[ 1 , 0 , 0 ]}_{G}^{T} ,$$

where R_{LG} is the transpose of R_{GL}.

In more detail, the gravity-based method can involve the following steps:
1. The smartphone and marker position data are resampled to a common sampling frequency (note that this step can be used for any of the synchronization methods described herein that rely on a comparison of wearable motion sensor data and reference motion sensing system data).
2. The aim of the method is to compare the reference system derived gravity (FIG. 9A, reference numeral 901) with the gravity direction estimated from the smartphone (FIG. 9A, reference 903) in the same reference frame. We chose the local reference frame (L, reference frame of the smartphone) for the comparison. Note that on FIG. 9A the vectors 903 and 901 appear to be aligned but they need not be and typically would not be aligned at all times as the smartphone moves with the subject. To transform the reference system gravity direction (901), we need to calculate the rotation matrix that aligns the reference frame M of the reference system with the local smartphone reference frame L (R_{LM}). This is calculated by computing an orthogonal vector basis using the three markers attached on the smartphone whose position is measured in the reference frame M by the motion capture system. The rotation matrix is calculated as a direction cosine matrix (DCM).
4. We know that acceleration and gyroscope are measured in the local reference frame L of the smartphone which coincides with the one defined by the markers (this coincidence is on purpose to simplify the calculations since we can choose how to define the orthogonal vector bases). We can use the acceleration and gyroscope readings to estimate the rotation matrix (R_{GL}) required to align one axis of the smartphone local reference frame L with the gravity direction using the Madwick filter. We call this reference frame smartphone gravity aligned (G). In this reference frame the gravity direction 903 is always aligned with one of the axes, in this case the x-axis (red). A Madwick filter tries to estimate a consistent reference frame in order to report the orientation of the smartphone. The Madgwick filter can be used with or without magnetometer data. When used without magnetometer as input the reference frame will be only aligned with gravity on one of the axes but not with the true magnetic north. This is sufficient for the present purpose as we only need to determine the gravity direction and do not care about where north is.
5. We can use the above to obtain the two gravity directions in the local reference frame of the smartphone L as follows:
   g_{mocap_L}=R_{LM}*[0, −1, 0] since gravity points along the negative y-axis (green) in the reference frame M; and
   g_{smartphone_L}=R_{LG}*[1, 0, 0]=R_{GL}^{T}*[1, 0, 0] since gravity points along the positive x-axis (red), in the reference frame G.

These two values can be compared by cross-correlation to align the signals.

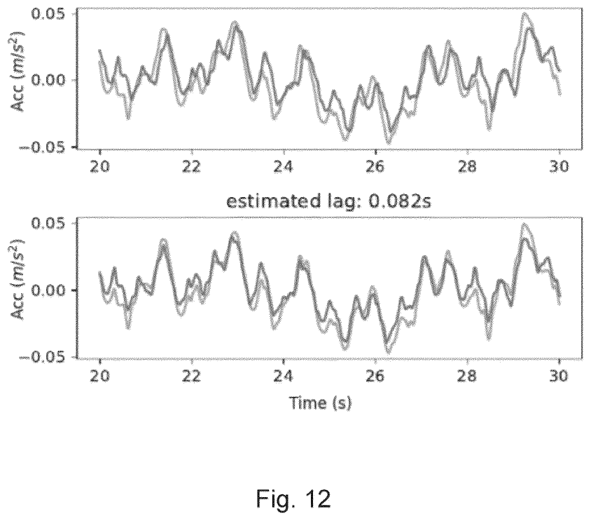

The anecdotal cross-correlation example depicted in FIG. 12 highlights a similar improvement with this correction method as previously observed with the acceleration-based method. However, this improvement was also observed during static balance tasks. Applying this gravity-based post-synchronization correction across all tests and study participants reduced the bias in the time points at which heel strike events were detected by the two measurement systems from an average (SD) 47.4 (35.6) ms to 11.2 (33.4) ms.

FIG. 10B shows the distribution of the lag between the acceleration of the smartphone and the acceleration calculated for the Vicon markers attached to the smartphone, calculated using the gravity-based method, across all study participants. This shows that the lag values were small and tightly for the Floodlight gait tests (2MWT and UTT) and for at least some of the SBT battery tests. Comparing the results of FIGS. 10B and 10A shows that the gravity and acceleration-based methods perform similarly well across the active tests.

### Example 3—Synchronization of Smartphone Inertial Measurement Unit and Motion-Capture Data (GRF Sensors)

In this example, the inventors used the approach described in Example 1 to analyse motion sensing data. As explained above, the inventors identified that inertia of the vibrating motor or delays introduced by the system used for creating the vibration pulse sequence may potentially lead to small, non-controlled lags between the smartphone and motion-capture sensor data. The inventors explored methods to address this, described in the present and previous examples.

### Force-Based Method

The force-based method is illustrated on FIG. 9C. It takes advantage of the fact that during walking or standing, the reaction forces applied on the feet have a direct effect on the participant's CoM [Uchida et al. 2021]. Assuming the participant's mass is concentrated in this location (indicated by the cross around the mid-section of the subject on FIG. 9C), this interaction can be approximated by writing the CoM equation of motion (Σf=ma). The forces applied to the feet (f1, f2 on FIG. 9C, first panel) can be virtually displaced and applied directly on the CoM (i.e., similar to an inverted pendulum model but simplifying the geometry, force “a” on FIG. 9C). The equation of motion can thus be simplified by taking the vector magnitudes, resulting in:

$$‖ {{f}_{1} ‖}_{2} + {‖ {f}_{2} ‖}_{2} = m * {‖ a ‖}_{2} ,$$

where f₁ and f₂ are the GRF of the left and right foot, respectively, m the participant's mass, and a the acceleration of the participant's CoM. The acceleration of the participant's CoM can be indirectly measured through the accelerometer readings of the smartphone at the waist level. The participant's mass is a constant and can therefore be ignored when performing a cross-correlation of the calculated forces with the GRF signal. By comparison, GRF can be directly measured with the force plates embedded in the treadmill. The smartphone CoM acceleration magnitude is then cross-correlated with the magnitude of the summed GRF to estimate the lag between the smartphone and motion-capture data after initial synchronization with the vibration-based method.

FIG. 9D shows an example of a calculate force obtained from the smartphone's accelerometer data (blue) and measured force from the GRF sensors (black) before (top) and after (bottom) synchronization by cross-correlation. This shows that the approximation above is valid as the calculated force (from the smartphone) at least correlates with the measured force (from the GRF sensors) sufficiently to be able to calculate a lag that can be used to synchronize the smartphone and GRF sensor data.

Similarly to what was described in Example 2, the detected lag was estimated across all Floodlight gait and balance tests with the force-based lag estimation method. The distributions of lags in each test are show on FIG. 10C. The detected lags were consistent with the lags detected with the acceleration-based method (Example 2) for the gait tests. The force-based method additionally showed very low variability on the SBT battery, and hence very high consistency, in the detected lag. This suggests that the force-based method is ideally suited for correcting the lag present in the SBT data as well as active test data (with or without initial synchronization with the vibration-based method).

### Example 4—Discussion

When comparing data collected with different measurement systems, it is critical that the data are correctly annotated and synchronized across the different systems. In these examples, the inventors presented new solutions for both challenges and illustrated their use on data collected with our smartphone sensor-based Floodlight technology and a motion-capture system.

The vibration pulse sequence generated by the motion-capture computer automatically encoded information on test type. This information was subsequently decoded to validate that the information matched between the two measurement systems, allowing correction of any erroneous entries. The vibration pulse sequence was also used for synchronizing the smartphones running the Floodlight technology with the motion-capture system (vibration-based method). However, close inspection revealed that the data collected with these two systems did not align perfectly with each other (FIG. 8). The initiation of the motion-capture recording by Motek software that interfaces with the Vicon motion-capture system and the initiation of the vibration command to the vibrating motor after pressing the start button are two separate processes. The time it takes for them to be realized is something that the inventors could not control in the present setup, thus resulting in small differences between the two onset events. Regardless of the underlying hardware/software cause, the lag can be estimated and subsequently corrected by the additionally proposed post-synchronization correction methods.

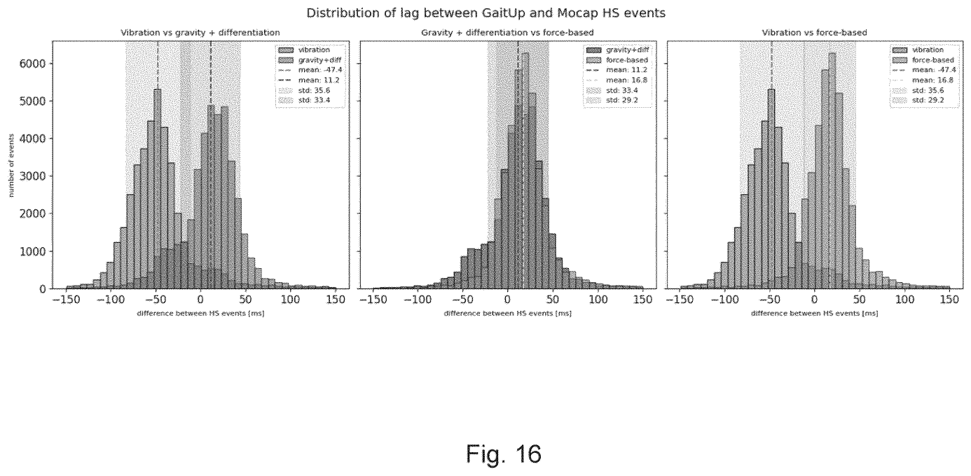

The inventors explored two different categories of methods to estimate the residual lag, which were all applied to each test execution after the initial synchronization with the vibration-based method. The acceleration-based method worked well for the 2MWT and UTT, leading to consistent lag values (FIG. 10B). During static balance tests it is possible that the participant's sway produces only small accelerations that are dominated by measurement noise making the detection of similarities through cross-correlation due to low signal-to-noise ratio challenging. Therefore, methods that are independent of the markers' acceleration may also be useful, particularly to synchronize smartphone and motion-capture data collected during static balance tasks. The gravity-based method, for example, involves comparing the unit gravitation direction of both measurements system. FIGS. 12 and 10C demonstrate that this method is also suited to correct the offset observed on at least some of the SBT data after the initial synchronization with the vibration-based method. The force-based method, for example, involves comparing the CoM acceleration estimated with the smartphones with the GRF measured with the treadmill. FIG. 10C demonstrates that this method can consistently estimate the lag present after the initial synchronization with the vibration-based method across all gait and balance test types. Similar results were obtained without the initial synchronization with the vibration-based method. Further, FIG. 16 shows that the methods described in these examples can be used to improve the comparison of gait features detected using the different sensors.

In these examples, the inventors illustrated the use of these solutions for synchronizing smartphone sensor data with motion-capture data, but these solutions are equally effective when applied to data collected with GaitUp sensors or other wearable devices.

In conclusion, the comparison of sensor data collected with different measurement systems in a gait and balance laboratory study poses several challenges relating to the annotation and synchronization of sensor data across different measurement systems. The inventors devised innovative solutions for both challenges. Automatic encoding and decoding of test type through a customized, binary vibration pulse sequence is a helpful tool for ensuring that this information matches across measurement systems. Additionally, this pulse sequence coupled with post-synchronization correction can be used for accurate synchronization of sensor data. The inventors further developed synchronization methods that can be used alone or after this initial synchronization (which is what was done in these examples, and the methods are therefore referred to as “post-synchronization methods” even though prior synchronization is advantageous but not required). The choice of the post-synchronization method may depend on the test performed by the study participants and on the test conditions. The force-based methods work well for both walking and static balance tasks, but requires specific sensors (e.g. force plates) to measure GRF. The gravity-based method works well for walking tasks, but is not as accurate as the force-based method so the force-based method may still be advantageous when GRF sensors are available. If they are not available, the simpler acceleration-based method is still well suited, particularly for walking tasks. The inventors further identified that it is computationally feasible in many cases to use a plurality of methods, and use for any particular data set the method that results in the best visual alignment and//or most concordant motion-derived metrics (e.g. gait features). In other words, as it is sometimes difficult to predict which method will be most suitable to synchronize a particular data set, a plurality of the methods described may be applied by default and the most suitable one for the particular data at hand may be selected.

### REFERENCES

All references cited herein are incorporated herein by reference in their entirety and for all purposes to the same extent as if each individual publication or patent or patent application was specifically and individually indicated to be incorporated by reference in its entirety.
Montalban, X., Graves, J., Midaglia, L., et al. A smartphone sensor-based digital outcome assessment of multiple sclerosis. Mult Scler J. 2022;28(4):654-664.
Kothe, C., Medine, D., Boulay, C. et al. Labstreaminglayer. https://labstreaminglayer.readthedocs.io/info/intro.html. 2019. [Accessed Dec. 13, 2022].
TimeServer. Time synchronization for Android via TimeServer. https://www.timeservers.net/sync/ntp/android. 2022. [Accessed Dec. 13, 2022].
Bourke A. K., Scotland A., Lipsmeier F., et al. Gait characteristics harvested during a smartphone-based self-administered 2-minute walk test in people with multiple sclerosis: test-retest reliability and minimum detectable change. Sensors. 2020;20(20):5906.
Cheng, W.-Y., Bourke, A. K., Lipsmeier, F., et al. U-turn speed is a valid and reliable smartphone-based measure of multiple sclerosis-related gait and balance impairment. Gait & Posture. 2021;84:120-126.
Uchida, T. K. and Delp, S. L. Biomechanics of Movement: The Science of Sports, Robotics, and Rehabilitation. MIT Press (1st edition). 2021.
Sebastian Madgwick. An efficient orientation filter for inertial and inertial/magnetic sensor arrays. Apr. 30, 2010. www.x-io.co.uk/open-source-imu-and-ahrs-algorithms
Lyons, Richard G. Understanding Digitla Signal Processing. Prentice Hall; 2nd edition (Jan. 1, 2004).

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The terms “about” or “approximately” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A computer-implemented method comprising:
providing data from a wearable motion sensing system worn by a subject and data from a reference motion sensing system recording the movement of the subject, wherein the reference motion sensing system is configured to record the position of one or more markers as a function of time and wherein the wearable motion sensing system is configured to record the acceleration and angular velocity of one or more wearable motion sensors comprised in the wearable motion sensing system worn by the subject; and
synchronizing the data from the wearable motion sensing system and the reference motion sensing system by:
determining a first signal derived from the acceleration and angular velocity of a sensor of the wearable motion sensing system, as a function of time;
determining a corresponding second signal derived from the position of one or more markers as measured by the reference motion sensing system, as a function of time; and
identifying a time lag that aligns the first signal and the second signal,
optionally wherein the first signal and second signal are both determined in a first reference frame.

2. wherein the wearable motion sensing system comprises one or more wearable motion sensors that each comprise an accelerometer and optionally a gyroscope; and/or
wherein the reference motion sensing system comprises a plurality of cameras located at different angles relative to the subject, optionally wherein the cameras are infrared cameras.

3. The method of any preceding claim, wherein the sensor of the wearable motion sensing system is associated with a plurality of markers detectable by the reference motion sensing system, and wherein the method comprises identifying the value of a first rotation matrix (R_{LM}, R_{ML}) as a function of time, wherein the first rotation matrix aligns the reference frames of the sensor of the wearable motion system (L) and the reference motion system (M), and wherein identifying the value of the first rotation matrix as a function of time comprises identifying an orthogonal vector basis associated with the sensor in the reference frame of the reference motion sensing system using the plurality of markers.

4. The method of any preceding claim, wherein synchronizing the data from the wearable motion sensing system and the reference motion sensing system comprises:
determining a first signal (g_{smartphoneL}(t)) corresponding to the unit gravity direction associated with a sensor of the wearable motion sensing system, as a function of time and in a first reference frame;
determining a second signal (g_{mocapL}(t)) corresponding to the unit gravity direction measured by the reference motion sensing system, as a function of time and in the first reference frame; and
identifying a time lag that aligns the first signal and the second signal,
optionally wherein the first reference frame is the local reference frame of the sensor of the wearable motion sensing system (L) and wherein determining the second signal comprises identifying the value of a first rotation matrix that aligns the reference frame of the reference motion sensing system with the first reference frame (R_{ML}), as a function of time, or wherein the first reference frame is the reference frame of the reference motion sensing system (M), and wherein determining the first signal comprises identifying the value of a first rotation matrix that aligns the local reference frame of the sensor with the first reference frame (R_{LM}), as a function of time.

5. The method of claim 4, wherein determining the first signal further comprises identifying the value of a second rotation matrix that aligns an axis of the local reference frame of the sensor with the gravitational axis (R_{GL}), as a function of time.

6. The method of any of claim 4 or 5, wherein determining the second signal comprises determining the value of the expression
$${R}_{LM} ( t ) * {[ {v}_{1 M} , {v}_{2 M} , {v}_{3 M} ]}_{M}^{T}$$
where R_{LM} (t) is the value of a first rotation matrix that aligns the reference frame of the reference motion sensing system with the first reference frame, as a function of time, and [v_{1M},v_{2M},v_{3M}]_{M} is the unit gravity vector in the reference frame of the reference motion sensing system, and/or wherein determining the first signal comprises determining the value of the expression
$${R}_{GL}^{T} ( t ) * {[ {v}_{1 G} , {v}_{2 G} , {v}_{3 G} ]}_{G}^{T}$$
where R_{GL}(t) is the value of a second rotation matrix that aligns an axis of the local reference frame of the sensor with the gravitational axis (R_{GL}), as a function of time, and [v_{1G},v_{2G},v_{3G}]_{G} is the unit gravity vector in the local reference frame of the sensor rotated to have an axis aligned with the gravitational axis.

7. The method of any of preceding claim, wherein synchronizing the data from the wearable motion sensing system and the reference motion sensing system comprises:
determining a first signal corresponding to the acceleration of a sensor of the wearable motion sensing system, as measured by the sensor and adjusted to remove the contribution of the acceleration of gravity, as a function of time and in a first reference frame;
determining a second signal corresponding to the acceleration of a marker associated with the sensor the wearable motion system as detected by the reference motion sensing system, as a function of time and in the first reference frame; and
identifying a time lag that aligns the first signal and the second signal.

8. The method of claim 7, wherein determining the second signal comprises determining the position of the marker as a function of time in the first reference frame and determining the second derivative of the determined position, optionally wherein determining the second signal comprises smoothing the position of the marker as a function of time.

9. The method of claim 8, wherein the first reference frame is the reference frame of the reference motion sensing system (M) and determining the position comprises using the position of the marker recorded by the reference motion sensing system, and/or wherein determining the first signal comprises:
identifying the value of a first rotation matrix (R_{LM}) as a function of time, wherein the first rotation matrix aligns the reference frame of the sensor of the wearable motion system (L) with the reference frame of the reference motion system (M), and
transforming the acceleration of the sensor of the wearable motion sensing system into the first reference frame using the first rotation matrix; and optionally removing the contribution of gravity from the transformed acceleration; or
wherein determining the first signal comprises: obtaining a linear acceleration from the sensor, and wherein the first signal is the magnitude of the linear acceleration from the sensor and the second signal is the magnitude of the second derivative of the determined position of a marker associated with the sensor as measured by the reference motion sensing system.

10. The method of any preceding claim, wherein identifying a time lag that aligns the first signal and the second signal is performed by determining the cross-correlation of the first and second signals and/or
wherein synchronizing the data from the wearable motion sensing system and the reference motion sensing system comprises identifying a time lag that aligns the first signal and the second signals as the time lag corresponding to maximum of the cross-correlation of the first and second signal, and optionally applying the identified time lag to the data from the wearable motion sensing system or the reference motion sensing system.

11. The method of any preceding claim, wherein the wearable motion sensing system comprises a plurality of wearable motion sensors and the method further comprises synchronising the plurality of wearable motion sensors based on a signal corresponding to a mechanical perturbation to which the plurality of wearable motion sensors were simultaneously exposed, in data series recorded by the plurality of wearable motion sensors.

12. The method of claim 11, wherein the signal corresponding to a mechanical perturbation comprises a signal at a first time point and a second time point, and wherein the synchronising the plurality of wearable motion sensors comprises:
identifying a first time lag for a sensor of the plurality of wearable motion sensor, optionally for each of the sensors individually, by aligning a portion of the data series of the sensor comprising the signal at the first time point with a portion of the data series of the reference motion sensor comprising the signal at the first time point; and
identifying a second time lag for a sensor of the plurality of wearable motion sensor, optionally for each of the sensors individually, by aligning a portion of the data series of the sensor comprising the signal at the second time point with a portion of the data series of the reference motion sensor comprising the signal at the second time point,
optionally wherein identifying a time lag by aligning the portions of the data series comprises performing a cross-correlation between the portions of the data series.

13. The method of any preceding claim, wherein the method further comprises:
(i) synchronising a sensor of the wearable motion system with the reference motion sensing system based on a signal corresponding to a mechanical perturbation to which the sensor was exposed in the data recorded by the sensor, wherein the signal is associated with a predetermined time in the reference motion sensing system, and/or
(ii) decoding metadata encoded in the data recorded by a sensor of the wearable motion sensing system using a signal in the data corresponding to a mechanical perturbation to which the sensor was exposed,
optionally wherein the method comprises exposing the sensor of the wearable motion system to the mechanical perturbation at the predetermined time, and/or wherein the predetermined time is the start of the recording from the reference motion sensing system and/or wherein the method comprises identifying a portion of a data series recorded by the sensor of the wearable motion system that comprises a signal corresponding to the mechanical perturbation, and/or wherein the method comprises detecting the onset of the signal corresponding to the mechanical perturbation in the data recorded by the sensor and optionally correcting a data series recorded by a sensor of the wearable motion system or a data series recorded by the reference motion sensing system using the time stamp of the identified onset or a difference between the time stamp of the identified onset and the time stamp of the predetermined time.

14. A computer-implemented method of analysing motion sensing data, the method comprising:
Performing the method of any preceding claim;
Comparing the synchronised data from the wearable motion sensing device and the reference motion sensing system to identify one or more motion features and/or clinically relevant parameters derived from the wearable motion sensor that are validated in the reference motion sensing system; and
optionally:
Obtaining motion sensing data associated with a subject from a wearable motion sensing system; and
Determining the value of one or more validated motion features, optionally comprising a gait feature, and/or clinically relevant parameters derived from the motion sensing data from the wearable motion sensing system.

15. A system comprising:
a processor; and
a computer readable medium comprising instructions that, when executed by the processor, cause the processor to perform the steps of the method of any of claims 1 to 14, optionally wherein the system further comprise one or more of: one or more wearable motion sensors, one or more motion marker tracking systems, one or more ground reaction force sensor systems, and one or more mechanical perturbation means.
